SEQUENCE LISTING (33 sequences)

SEQ ID NO: 1 - DNA, 1495 bp, Homo sapiens
ctctcctttcccgaacctcccggggtgcagcctagagccctcccgcgcggctgactccagagtagaggaagggaggcggcctccggctggtcccccgaagccctcgctgccccgcagatgcggatggccagccagtagcgggcggtggccccgcgtcccgggagcgcacagcaatgcaggcgcttaacattaccccggagcagttctctcggctgctgcgggaccacaacctgacgcgggagcagttcatcgctctgtaccggctgcgaccgctcgtctacaccccagagctgccgggacgcgccaagctggccctcgtgctcaccggcgtgctcatcttcgccctggcgctctttggcaatgctctggtgttctacgtggtgacccgcagcaaggccatgcgcaccgtcaccaacatctttatctgctccttggcgctcagtgacctgctcatcaccttcttctgcattcccgtcaccatgctccagaacatttccgacaactggctggggggtgctttcatttgcaagatggtgccatttgtccagtctaccgctgttgtgacagaaatcctcactatgacctgcattgctgtggaaaggcaccagggacttgtgcatccttttaaaatgaagtggcaatacaccaaccgaagggctttcacaatgctaggtgtggtctggctggtggcagtcatcgtaggatcacccatgtggcacgtgcaacaacttgagatcaaatatgacttcctatatgaaaaggaacacatctgctgcttagaagagtggaccagccctgtgcaccagaagatctacaccaccttcatccttgtcatcctcttcctcctgcctcttatggtgatgcttattctgtacagtaaaattggttatgaactttggataaagaaaagagttggggatggttcagtgcttcgaactattcatggaaaagaaatgtccaaaatagccaggaagaagaaacgagctgtcattatgatggtgacagtggtggctctctttgctgtgtgctgggcaccattccatgttgtccatatgatgattgaatacagtaattttgaaaaggaatatgatgatgtcacaatcaagatgatttttgctatcgtgcaaattattggattttccaactccatctgtaatcccattgtctatgcatttatgaatgaaaacttcaaaaaaaatgttttgtctgcagtttgttattgcatagtaaataaaaccttctctccagcacaaaggcatggaaattcaggaattacaatgatgcggaagaaagcaaagttttccctcagagagaatccagtggaggaaaccaaaggagaagcattcagtgatggcaacattgaagtcaaattgtgtgaacagacagaggagaagaaaaagctcaaacgacatcttgctctctttaggtctgaactggctgagaattctcctttagacagtgggcattaattataacaatatcttcataattaatg

SEQ ID NO: 2 - protein, 415 aa, Homo sapiens
MQALNITPEQFSRLLRDHNLTREQFIALYRLRVLTGVLIFALALFGNALVFYVVTRSKAMRTVTNIFICSLALSDLLITFFCIPVTMLQNISDNWLGGAFICKMVPFVQSTAVVTEILTMTCIAVERHQGLVHPFKMKWQYTNRRAFTMLGVVWLVAVIVGSPMWHVQQLEIKYDFLYEKEHICCLEEWTSPVHQKIYTTFILVILFLLPLMVMLILYSKIGYELWIKKRVGDGSVLRTIHGKEMSKIARKKKRAVIMMVTVVALFAVCWAPFHVVHMMIEYSNFEKEYDDVTIKMIFAIVQIIGFSNSICNPIVYAFMNENFKKNVLSAVCYCIVNKTFSPAQRHGNSGITMMRKKAKFSLRENPVEETKGEAFSDGNIEVKLCEQTEEKKKLKRHLALFRSELAENSPLDSGH

SEQ ID NO: 3 - DNA, 27 bp, artificial sequence
  primer directed to human SNORF 44 receptor
ccatcctaatacgactcactatagggc

SEQ ID NO: 4 - DNA, 23 bp, artificial sequence
  primer directed to human SNORF 44 receptor
actcactatagggctcgagcggc

SEQ ID NO: 5 - DNA, 25 bp, artificial sequence
  primer directed to human SNORF 44 receptor
ttatgcttccggctcgtatgttgtg

SEQ ID NO: 6 - DNA, 26 bp, artificial sequence
  primer directed to human SNORF 44 receptor
atgtgctgcaaggcgattaagttggg

SEQ ID NO: 7 - DNA, 24 bp, artificial sequence
  primer directed to human SNORF 44 receptor
gtngbnntnttynynntnwgytgg

SEQ ID NO: 8 - DNA, 24 bp, artificial sequence
  primer directed to human SNORF 44 receptor
narraangcrtanadnannggrtt

SEQ ID NO: 9 - DNA, 27 bp, artificial sequence
  primer directed to human SNORF 44 receptor
ttcattcataaatgcatagacaatggg

SEQ ID NO: 10 - DNA, 26 bp, artificial sequence
  primer directed to human SNORF 44 receptor
caggtgctttcatttgcaagatggtg

SEQ ID NO: 11 - DNA, 26 bp, artificial sequence
  primer directed to human SNORF 44 receptor
caatctgaagggcattaattatgaag

SEQ ID NO: 12 - DNA, 26 bp, artificial sequence
  primer directed to human SNORF 44 receptor
caccattccatgttgtccatatgatg

SEQ ID NO: 13 - DNA, 27 bp, artificial sequence
  primer directed to human SNORF 44 receptor
gtacagaataagcatcaccataagagg

SEQ ID NO: 14 - DNA, 28 bp, artificial sequence
  primer directed to human SNORF 44 receptor
ctcttatggtgatgcttattctgtacag

SEQ ID NO: 15 - DNA, 26 bp, artificial sequence
  primer directed to human SNORF 44 receptor
tgcacaagtccctggtgcctttccac

SEQ ID NO: 16 - DNA, 26 bp, artificial sequence
  primer directed to human SNORF 44 receptor
gcaatgcaggtcatagtgaggatttc

SEQ ID NO: 17 - DNA, 27 bp, artificial sequence
  primer directed to human SNORF 44 receptor
cacaacagcggtagactggacaaatgg

SEQ ID NO: 18 - DNA, 26 bp, artificial sequence
  primer directed to human SNORF 44 receptor
ggagcagataaagatgttggtgacgg

SEQ ID NO: 19 - DNA, 25 bp, artificial sequence
  primer directed to human SNORF 44 receptor
ccacgtagaacaccagagcattgcc

SEQ ID NO: 20 - DNA, 25 bp, artificial sequence
  primer directed to human SNORF 44 receptor
ccggtgagcacgagggccagcttgg

SEQ ID NO: 21 - DNA, 37 bp, artificial sequence
  primer directed to human SNORF 44 receptor
atctatggatcctatcgtgggtttgatccctgagctg

SEQ ID NO: 22 - DNA, 38 bp, artificial sequence
  primer directed to human SNORF 44 receptor
atctataagcttttctctttgggttacaatctgaaggg

SEQ ID NO: 23 - DNA, 27 bp, artificial sequence
  primer directed to human SNORF 44 receptor
tcaccaacatctttatctgctccttgg

SEQ ID NO: 24 - DNA, 27 bp, artificial sequence
  primer directed to human SNORF 44 receptor
cagcaatgcaggcgcttaacattaccc

SEQ ID NO: 25 - DNA, 20 bp, artificial sequence
  primer directed to human SNORF 44 receptor
taatacgactcactataggg

SEQ ID NO: 26 - DNA, 16 bp, artificial sequence
  primer directed to human SNORF 44 receptor
caggaaacagctatga

SEQ ID NO: 27 - DNA, 18 bp, artificial sequence
  primer directed to human SNORF 44 receptor
cgtgctcatcttcgccct

SEQ ID NO: 28 - DNA, 16 bp, artificial sequence
  primer directed to human SNORF 44 receptor
tgcgcatggccttgct

SEQ ID NO: 29 - DNA, 28 bp, artificial sequence
  probe directed to human SNORF 44 receptor
cgctctttggcaatgctctggtgttcta

SEQ ID NO: 30 - protein, 8 aa, Homo sapiens
YSMEHFRW

SEQ ID NO: 31 - protein, 6 aa, Homo sapiens
MGHFRF

SEQ ID NO: 32 - protein, 7 aa, Homo sapiens
YGGHFRW

SEQ ID NO: 33 - protein, 8 aa, Homo sapiens
  MISC_FEATURE (1)..(2): x = any amino acid present or absent
XXXXHFRX

(19) United States
(12) Patent Application Publication
(10) Pub. No.: US 2003/0143670 A1
(43) Pub. Date: Jul. 31, 2003

(54) DNA encoding SNORF44 receptor

(76) Inventors: James A. Bonini, Oakland, NJ (US); Ling-Yan Huang, Sunnyvale, CA (US); Amy E. Wilson, Woodstock, NY (US)

Correspondence Address: John P. White, Cooper & Dunham LLP, 1185 Avenue of the Americas, New York, NY, 10036, US

(21) Appl. No.: 10/080,263

(22) Filed: Feb. 20, 2002

Related U.S. Application Data
(63) Continuation-in-part of application No. 09/321,683, filed on May 28, 1999, now abandoned.

(51) Int. Cl. C12P 21/02; C12N 5/06; C07K 14/705; C07H 21/04

(52) U.S. Cl. 435/69.1; 435/320.1; 435/325; 530/350; 536/23.5

(57) ABSTRACT

This invention provides isolated nucleic acids encoding mammalian SNORF44 receptor, purified mammalian SNORF44 receptor, vectors comprising nucleic acid encoding mammalian SNORF44 receptor, cells comprising such vectors, antibodies directed to mammalian SNORF44 receptor, nucleic acid probes useful for detecting nucleic acid encoding mammalian SNORF44 receptor, antisense oligonucleotides complementary to unique sequences of nucleic acid encoding mammalian SNORF44 receptor, transgenic, nonhuman animals which express DNA encoding normal or mutant mammalian SNORF44 receptor, and methods of isolating mammalian SNORF44 receptor. This invention also provides methods of treating an abnormality that is linked to the activity of a mammalian SNORF44 receptor, as well as methods of determining binding of compounds to a mammalian SNORF44 receptor, methods of identifying agonists and antagonists of a SNORF44 receptor, and agonists and antagonists so identified.

This application is a continuation-in-part of U.S. Ser. No. 09/321,683, filed May 28, 1999, the contents of which are hereby incorporated by reference into the subject application.

### BACKGROUND OF THE INVENTION

Throughout this application various publications are referred to by partial citations within parenthesis. Full citations for these publications may be found at the end of the specification immediately preceding the claims. The disclosures of these publications, in their entireties, are hereby incorporated by reference into this application in order to more fully describe the state of the art to which the invention pertains.

Neuroregulators comprise a diverse group of natural products that subserve or modulate communication in the nervous system. They include, but are not limited to, neuropeptides, amino acids, biogenic amines, lipids and lipid metabolites, and other metabolic byproducts. Many of these neuroregulator substances interact with specific cell surface receptors which transduce signals from the outside to the inside of the cell. G-protein coupled receptors (GPCRs) represent a major class of cell surface receptors with which many neurotransmitters interact to mediate their effects. GPCRs are characterized by seven membrane-spanning domains and are coupled to their effectors via G-proteins linking receptor activation with intracellular biochemical sequelae such as stimulation of adenylyl cyclase. While structural motifs that characterize a GPCR can be recognized in the predicted amino acid sequence of a novel receptor, an endogenous ligand that activates the GPCR cannot always be predicted from its primary structure. Thus, a novel receptor sequence may be designated as an orphan GPCR when it possesses the structural motif characteristic of a G-protein coupled receptor, but an endogenous ligand for it has not yet been defined. Orphan receptors can also be activated by ligands other than an endogenous ligand. The identification of activating ligands which mimic the effects of an endogenous ligand allows both the characterization of the receptor signal transduction pathway and the development of methods for screening for compounds (agonists or antagonists) that activate or block receptor function. Such agonists or antagonists permit determination the biochemical role of the receptor in normal and pathological states, and thus therapeutic potential of drugs that would act at the receptor. Finally, the use of such agonists accelerates the discovery of an endogenous ligand through iterative structure/function analyses and datamining. This application describes the identification of three peptide agonists for the orphan GPCR designated SNORF44, and further describes their use in probing the functional activation of SNORF44 and in screening for compounds acting at SNORF44.

### SUMMARY OF THE INVENTION

This invention provides an isolated nucleic acid encoding a mammalian SNORF44 receptor.

This invention further provides a purified mammalian SNORF44 receptor protein.

Furthermore, this invention provides a nucleic acid probe comprising at least 15 nucleotides, which probe specifically hybridizes with a nucleic acid encoding a mammalian SNORF44 receptor, wherein the probe has a sequence complementary to a unique sequence present within one of the two strands of the nucleic acid encoding the human SNORF44 receptor contained in plasmid pcDNA. 1-hSNORF44-f (ATCC Patent Deposit Designation PTA-100).

This invention provides a nucleic acid probe comprising at least 15 nucleotides, which probe specifically hybridizes with a nucleic acid encoding a mammalian SNORF44 receptor, wherein the probe has a sequence complementary to a unique sequence present within (a) the nucleic acid sequence shown in FIGS. 1A-1C (SEQ ID NO: 1) or (b) the reverse complement thereof.

This invention further provides a transgenic, nonhuman mammal comprising a homologous recombination knockout of the native mammalian SNORF44 receptor.

This invention additionally provides a process for identifying a chemical compound which specifically binds to a mammalian SNORF44 receptor which comprises contacting cells containing DNA encoding, and expressing on their cell surface, the mammalian SNORF44 receptor, wherein such cells do not normally express the mammalian SNORF44 receptor, with the compound under conditions suitable for binding, and detecting specific binding of the chemical compound to the mammalian SNORF44 receptor.

Furthermore, this invention provides a process for identifying a chemical compound which specifically binds to a mammalian SNORF44 receptor which comprises contacting a membrane preparation from cells containing DNA encoding, and expressing on their cell surface, the mammalian SNORF44 receptor, wherein such cells do not normally express the mammalian SNORF44 receptor, with the compound under conditions suitable for binding, and detecting specific binding of the chemical compound to the mammalian SNORF44 receptor.

Moreover, this invention provides a process involving competitive binding for identifying a chemical compound which specifically binds to a mammalian SNORF44 receptor which comprises separately contacting cells expressing on their cell surface the mammalian SNORF44 receptor, wherein such cells do not normally express the mammalian SNORF44 receptor, with both the chemical compound and a second chemical compound known to bind to the receptor, and with only the second chemical compound, under conditions suitable for binding of such compounds to the receptor, and detecting specific binding of the chemical compound to the mammalian SNORF44 receptor, a decrease in the binding of the second chemical compound to the mammalian SNORF44 receptor in the presence of the chemical compound being tested indicating that such chemical compound binds to the mammalian SNORF44 receptor.

This invention also provides a process involving competitive binding for identifying a chemical compound which specifically binds to a mammalian SNORF44 receptor which comprises separately contacting a membrane preparation from cells expressing on their cell surface the mammalian SNORF44 receptor, wherein such cells do not normally express the mammalian SNORF44 receptor, with both the chemical compound and a second chemical compound known to bind to the receptor, and with only the second chemical compound, under conditions suitable for binding of such compounds to the receptor, and detecting specific binding of the chemical compound to the mammalian SNORF44 receptor, a decrease in the binding of the second chemical compound to the mammalian SNORF44 receptor in the presence of the chemical compound being tested indicating that such chemical compound binds to the mammalian SNORF44 receptor.

This invention further provides a method of screening a plurality of chemical compounds not known to bind to a mammalian SNORF44 receptor to identify a compound which specifically binds to the mammalian SNORF44 receptor, which comprises (a) contacting cells transfected with, and expressing, DNA encoding the mammalian SNORF44 receptor with a compound known to bind specifically to the mammalian SNORF44 receptor; (b) contacting the cells of step (a) with the plurality of compounds not known to bind specifically to the mammalian SNORF44 receptor, under conditions permitting binding of compounds known to bind to the mammalian SNORF44 receptor; (c) determining whether the binding of the compound known to bind to the mammalian SNORF44 receptor is reduced in the presence of the plurality of compounds, relative to the binding of the compound in the absence of the plurality of compounds; and if so (d) separately determining the binding to the mammalian SNORF44 receptor of each compound included in the plurality of compounds, so as to thereby identify any compound included therein which specifically binds to the mammalian SNORF44 receptor.

This invention still further provides a method of screening a plurality of chemical compounds not known to bind to a mammalian SNORF44 receptor to identify a compound which specifically binds to the mammalian SNORF44 receptor, which comprises (a) contacting a membrane preparation from cells transfected with, and expressing, DNA encoding the mammalian SNORF44 receptor with the plurality of compounds not known to bind specifically to the mammalian SNORF44 receptor under conditions permitting binding of compounds known to bind to the mammalian SNORF44 receptor; (b) determining whether the binding of a compound known to bind to the mammalian SNORF44 receptor is reduced in the presence of the plurality of compounds, relative to the binding of the compound in the absence of the plurality of compounds; and if so (c) separately determining the binding to the mammalian SNORF44 receptor of each compound included in the plurality of compounds, so as to thereby identify any compound included therein which specifically binds to the mammalian SNORF44 receptor.

Furthermore, this invention provides a method for diagnosing a predisposition to a disorder associated with the activity of a specific mammalian allele which comprises: (a) obtaining DNA of subjects suffering from the disorder; (b) performing a restriction digest of the DNA with a panel of restriction enzymes; (c) electrophoretically separating the resulting DNA fragments on a sizing gel; (d) contacting the resulting gel with a nucleic acid probe capable of specifically hybridizing with a unique sequence included within the sequence of a nucleic acid molecule encoding a mammalian SNORF44 receptor and labeled with a detectable marker; (e) detecting labeled bands which have hybridized to the DNA encoding a mammalian SNORF44 receptor of claim 1 to create a unique band pattern specific to the DNA of subjects suffering from the disorder; (f) repeating steps (a)-(e) with DNA obtained for diagnosis from subjects not yet suffering from the disorder; and (g) comparing the unique band pattern specific to the DNA of subjects suffering from the disorder from step (e) with the band pattern from step (f) for subjects not yet suffering from the disorder so as to determine whether the patterns are the same or different and thereby diagnose predisposition to the disorder if the patterns are the same.

This invention also provides a process for determining whether a chemical compound is a mammalian SNORF44 receptor agonist which comprises contacting cells transfected with and expressing DNA encoding the mammalian SNORF44 receptor with the compound under conditions permitting the activation of the mammalian SNORF44 receptor, and detecting any increase in mammalian SNORF44 receptor activity, so as to thereby determine whether the compound is a mammalian SNORF44 receptor agonist.

This invention further provides a process for determining whether a chemical compound is a mammalian SNORF44 receptor antagonist which comprises contacting cells transfected with and expressing DNA encoding the mammalian SNORF44 receptor with the compound in the presence of a known mammalian SNORF44 receptor agonist, under conditions permitting the activation of the mammalian SNORF44 receptor, and detecting any decrease in mammalian SNORF44 receptor activity, so as to thereby determine whether the compound is a mammalian SNORF44 receptor antagonist.

Moreover, this invention provides a process for determining whether a chemical compound specifically binds to and activates a mammalian SNORF44 receptor, which comprises contacting cells producing a second messenger response and expressing on their cell surface the mammalian SNORF44 receptor, wherein such cells do not normally express the mammalian SNORF44 receptor, with the chemical compound under conditions suitable for activation of the mammalian SNORF44 receptor, and measuring the second messenger response in the presence and in the absence of the chemical compound, a change in the second messenger response in the presence of the chemical compound indicating that the compound activates the mammalian SNORF44 receptor.

This invention further provides a process for determining whether a chemical compound specifically binds to and inhibits activation of a mammalian SNORF44 receptor, which comprises separately contacting cells producing a second messenger response and expressing on their cell surface the mammalian SNORF44 receptor, wherein such cells do not normally express the mammalian SNORF44 receptor, with both the chemical compound and a second chemical compound known to activate the mammalian SNORF44 receptor, and with only the second chemical compound, under conditions suitable for activation of the mammalian SNORF44 receptor, and measuring the second messenger response in the presence of only the second chemical compound and in the presence of both the second chemical compound and the chemical compound, a smaller change in the second messenger response in the presence of both the chemical compound and the second chemical compound than in the presence of only the second chemical compound indicating that the chemical compound inhibits activation of the mammalian SNORF44 receptor.

This invention provides a method of screening a plurality of chemical compounds not known to activate a mammalian SNORF44 receptor to identify a compound which activates the mammalian SNORF44 receptor which comprises: (a) contacting cells transfected with and expressing the mammalian SNORF44 receptor with the plurality of compounds not known to activate the mammalian SNORF44 receptor, under conditions permitting activation of the mammalian SNORF44 receptor; (b) determining whether the activity of the mammalian SNORF44 receptor is increased in the presence of one or more of the compounds; and if so (c) separately determining whether the activation of the mammalian SNORF44 receptor is increased by any compound included in the plurality of compounds, so as to thereby identify each compound which activates the mammalian SNORF44 receptor.

This invention further provides a method of screening a plurality of chemical compounds not known to inhibit the activation of a mammalian SNORF44 receptor to identify a compound which inhibits the activation of the mammalian SNORF44 receptor, which comprises: (a) contacting cells transfected with and expressing the mammalian SNORF44 receptor with the plurality of compounds in the presence of a known mammalian SNORF44 receptor agonist, under conditions permitting activation of the mammalian SNORF44 receptor; (b) determining whether the extent or amount of activation of the mammalian SNORF44 receptor is reduced in the presence of one or more of the compounds, relative to the extent or amount of activation of the mammalian SNORF44 receptor in the absence of such one or more compounds; and if so (c) separately determining whether each such compound inhibits activation of the mammalian SNORF44 receptor for each compound included in the plurality of compounds, so as to thereby identify any compound included in such plurality of compounds which inhibits the activation of the mammalian SNORF44 receptor.

This invention additionally provides a method of treating an abnormality in a subject wherein the abnormality is alleviated by increasing the activity of a mammalian SNORF44 receptor which comprises administering to the subject a compound which is a mammalian SNORF44 receptor agonist in an amount effective to treat the abnormality.

This invention further provides a method of treating an abnormality in a subject wherein the abnormality is alleviated by decreasing the activity of a mammalian SNORF44 receptor which comprises administering to the subject a compound which is a mammalian SNORF44 receptor antagonist in an amount effective to treat the abnormality.

### BRIEF DESCRIPTION OF THE FIGURES

FIGS. 1A-1C

Nucleotide sequence including sequence encoding a human SNORF44 receptor (SEQ ID NO: 1). The putative open reading frame (the shortest open reading frame) is indicated by underlining the start (ATG) codon (at positions 174-176) and the stop codon (at positions 1467-1469). In addition, partial 5′ and 3′ untranslated sequences are shown.

FIGS. 2A-2B

Deduced amino acid sequence (SEQ ID NO: 2) of the human SNORF44 receptor encoded by the longest open reading frame indicated in the nucleotide sequence shown in FIGS. 1A-1C (SEQ ID NO: 1). The seven putative transmembrane (TM) regions are underlined.

FIG. 3

Concentration-dependent stimulation of intracellular Ca²⁺ release by Peptide 1 in hSNORF44- and mock-transfected Cos-7 cells. The cells were loaded with fluo-3, a calcium indicator dye, for 1 h. Free intracellular calcium was measured at different time points using Fluorescence Imaging Plate Reader (FLIPR).

FIGS. 4A-4C

Concentration-dependent stimulation of inositol phosphate (IP) second messenger release by A) Peptide 1, B) Peptide 2, and C) Peptide 3 in hSNORF44-transfected and mock-transfected Cos-7 cells.

FIGS. 5A-5B

Peptide 1-induced calcium-activated chloride currents in Xenopus laevis oocytes expressing hSNORF44.

A) Electrophysiological response to Peptide 1 from voltage clamped oocyte injected with mRNA encoding hSNORF44. Trace is representative of 10 experiments carried out with similar results.

B) Uninjected oocyte (not expressing hSNORF44) does not respond to Peptide 1 (representative of 5 separate experiments).

### DETAILED DESCRIPTION OF THE INVENTION

This invention provides a recombinant nucleic acid comprising a nucleic acid encoding a mammalian SNORF44 receptor, wherein the mammalian receptor-encoding nucleic acid hybridizes under high stringency conditions to a nucleic acid encoding a human SNORF44 receptor and having a sequence identical to the sequence of the human SNORF44 receptor-encoding nucleic acid contained in plasmid pcDNA3.1-hSNORF44-f (ATCC Patent Deposit Designation PTA-100).

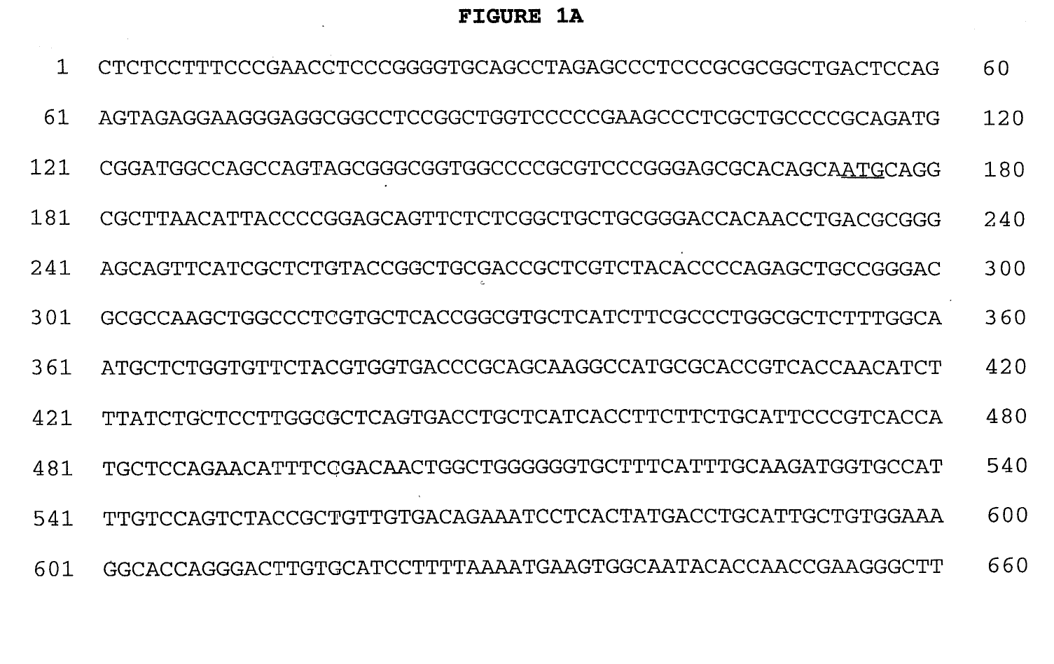

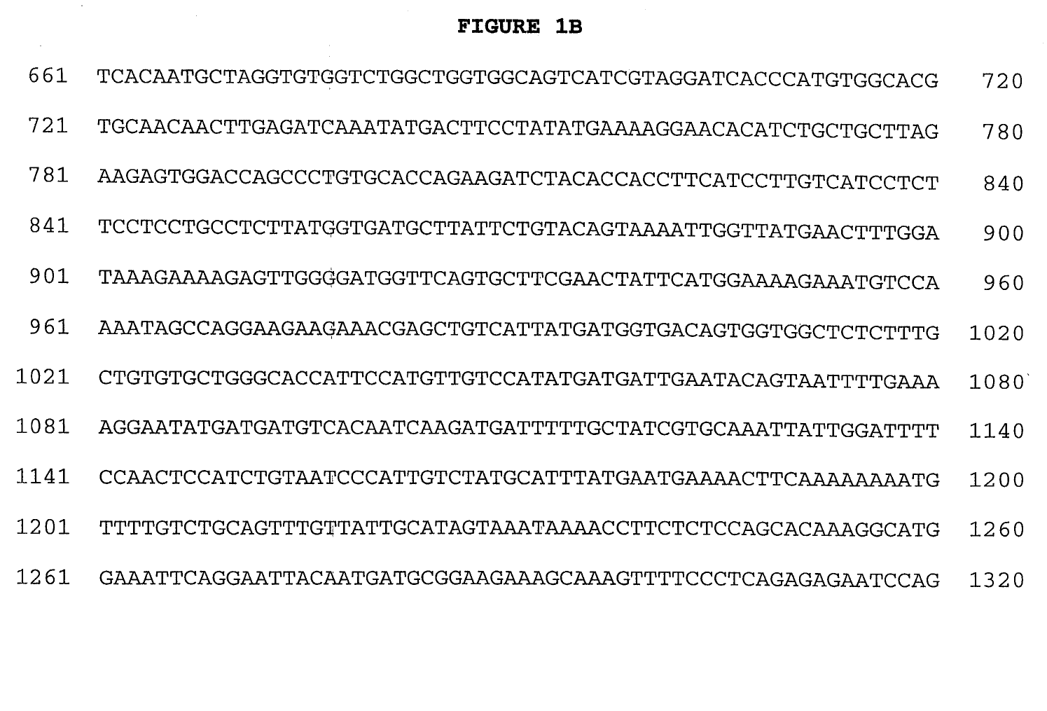

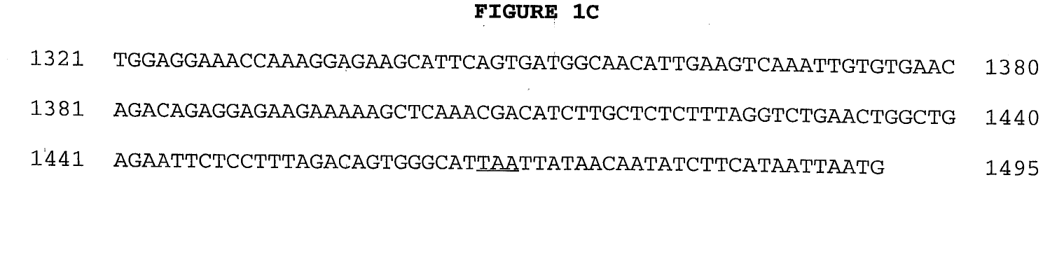

This invention further provides a recombinant nucleic acid comprising a nucleic acid encoding a human SNORF44 receptor, wherein the human SNORF44 receptor comprises an amino acid sequence identical to the sequence of the human SNORF44 receptor encoded by the longest open reading frame indicated in FIGS. 1A-1C (SEQ ID NO: 1).

The plasmid pEXJ.T3T7-hSNORF44-f was deposited on May 21, 1999, with the American Type Culture Collection (ATCC), 10801 University Blvd., Manassas, Va. 20110-2209, U.S.A. under the provisions of the Budapest Treaty for the International Recognition of the Deposit of Microorganisms for the Purposes of Patent Procedure and was accorded ATCC Patent Deposit Designation PTA-100.

This invention also contemplates recombinant nucleic acids which comprise nucleic acids encoding naturally occurring allelic variants of the mammalian SNORF44 receptors described above.

Hybridization methods are well known to those of skill in the art. For purposes of this invention, hybridization under high stringency conditions means hybridization performed at 40° C. in a hybridization buffer containing 50% formamide, 5×SSC, 7 mM Tris_{,} 1×Denhardt's, 25 μg/ml salmon sperm DNA; wash at 50° C. in 0.133 SSC, 0.1%SDS.

Throughout this application, the following standard abbreviations are used to indicate specific nucleotide bases:

A=adenine

G=guanine

C=cytosine

T=thymine

M=adenine or cytosine

R=adenine or guanine

W=adenine or thymine

S=cytosine or guanine

Y=cytosine or thymine

K=guanine or thymine

V=adenine, cytosine, or guanine (not thymine)

H=adenine, cytosine, or thymine (not cytosine)

B=cytosine, guanine, or thymine (not adenine)

N=adenine, cytosine, guanine, or thymine (or other modified base such as inosine)

I=inosine

Throughout this application, the following standard abbreviations are used to indicate specific amino acids:

| 3-character | | 1-character |
|---|---|---|
| abbreviation | Amino Acid | abbreviation |
| Ala | Alanine | A |
| Arg | Arginine | R |
| Asn | Asparagine | N |
| Asp | Aspartic Acid | D |
| Cys | Cysteine | C |
| Gln | Glutamine | Q |
| Glu | Glutamic Acid | E |
| Gly | Glycine | G |
| His | Histidine | H |
| Ile | Isoleucine | I |
| Leu | Leucine | L |
| Lys | Lysine | K |
| Met | Methionine | M |
| Phe | Phenylalanine | F |
| Pro | Proline | P |
| Ser | Serine | S |
| Thr | Threonine | T |
| Trp | Tryptophane | W |
| Tyr | Tyrosine | Y |
| Val | Valine | V |
| Asx | Asparagine/ | B |
| | Aspartic Acid | |
| Glx | Glutamine/ | Z |
| | Glutamic Acid | |
| *** | (End) | * |
| Xxx | Any amino acid or as | X |
| | specified | |

Furthermore, the term “agonist” is used throughout this application to indicate any peptide or non-peptidyl compound which increases the activity of any of the polypeptides of the subject invention. The term “antagonist” is used throughout this application to indicate any peptide or non-peptidyl compound which decreases the activity of any of the polypeptides of the subject invention.

Furthermore, as used herein, the phrase “pharmaceutically acceptable carrier” means any of the standard pharmaceutically acceptable carriers. Examples include, but are not limited to, phosphate buffered saline, physiological saline, water, and emulsions, such as oil/water emulsions.

It is possible that the mammalian SNORF44 receptor gene contains introns and furthermore, the possibility exists that additional introns could exist in coding or non-coding regions. In addition, spliced form(s) of mRNA may encode additional amino acids either upstream of the currently defined starting methionine or within the coding region. Further, the existence and use of alternative exons is possible, whereby the mRNA may encode different amino acids within the region comprising the exon. In addition, single amino acid substitutions may arise via the mechanism of RNA editing such that the amino acid sequence of the expressed protein is different than that encoded by the original gene. (Burns, C. M. et al., 1997; Chu, et al., 1996). Such variants may exhibit pharmacologic properties differing from the polypeptide encoded by the original gene.

This invention provides splice variants of the mammalian SNORF44 receptor disclosed herein. This invention further provides for alternate translation initiation sites and alternately spliced or edited variants of nucleic acids encoding the mammalian SNORF44 receptor of this invention.

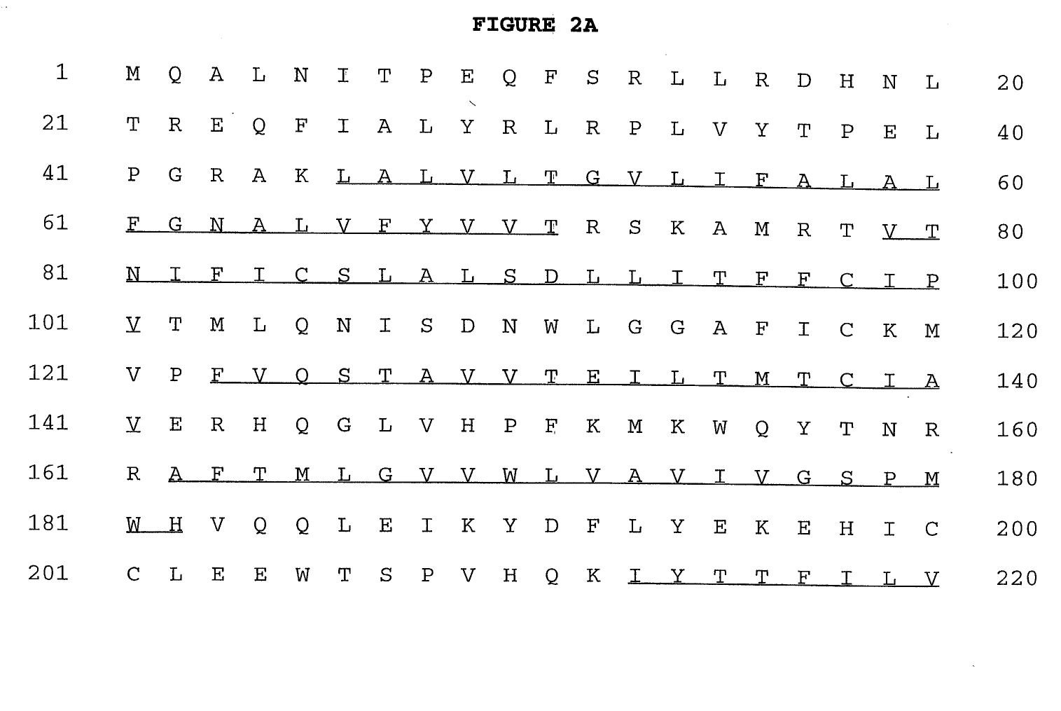

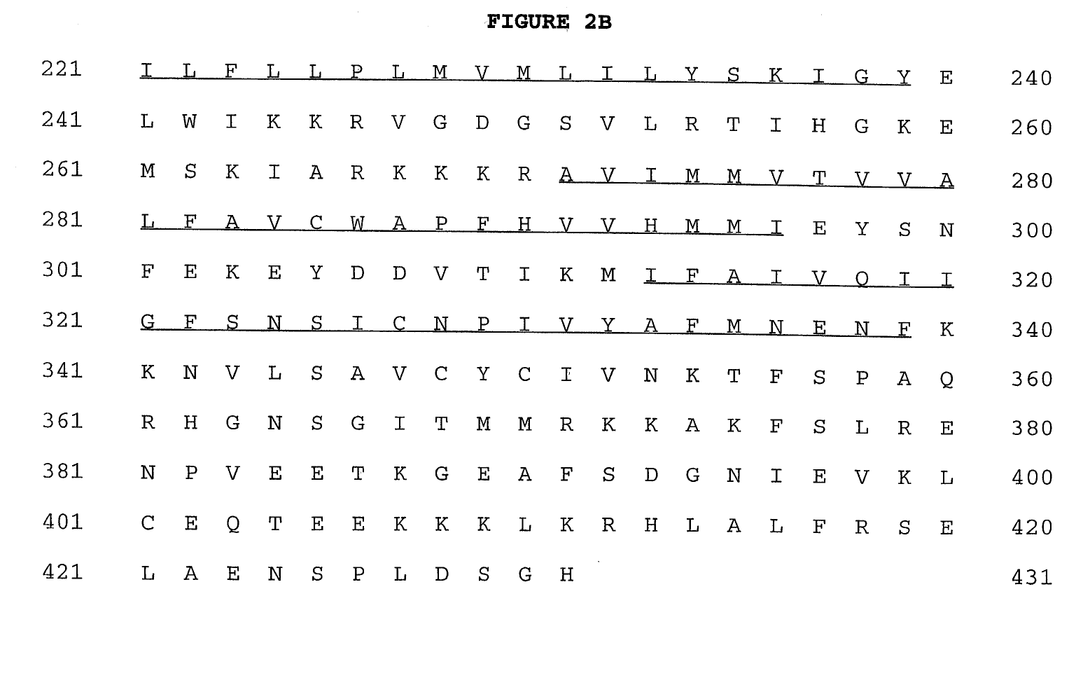

The nucleic acids of the subject invention also include nucleic acid analogs of the human SNORF44 receptor gene, wherein the human SNORF44 receptor gene comprises the nucleic acid sequence shown in FIGS. 1A-1C (SEQ ID NO: 1) or contained in plasmid pcDNA3.1-hSNORF44-f (ATCC Patent Deposit Designation PTA-100). Nucleic acid analogs of the human SNORF44 receptor genes differ from the human SNORF44 receptor genes described herein in terms of the identity or location of one or more nucleic acid bases (deletion analogs containing less than all of the nucleic acid bases shown in FIGS. 1A-1C or contained in plasmid pcDNA3.1-hSNORF44-f, substitution analogs wherein one or more nucleic acid bases shown in FIGS. 1A-1C or contained in plasmid pcDNA3.1-hSNORF44-f (ATCC Patent Deposit Designation PTA-100), are replaced by other nucleic acid bases, and addition analogs, wherein one or more nucleic acid bases are added to a terminal or medial portion of the nucleic acid sequence) and which encode proteins which share some or all of the properties of the proteins encoded by the nucleic acid sequences shown in FIGS. 1A-1C or contained in plasmid pcDNA3.1-hSNORF44-f (ATCC Patent Deposit Designation PTA-100). In one embodiment of the present invention, the nucleic acid analog encodes a protein which has an amino acid sequence identical to that shown in FIGS. 2A-2B or encoded by the nucleic acid sequence contained in plasmid pcDNA3.1-hSNORF44-f (ATCC Patent Deposit Designation PTA-100). In another embodiment, the nucleic acid analog encodes a protein having an amino acid sequence which differs from the amino acid sequences shown in FIGS. 2A-2B or encoded by the nucleic acid contained in plasmid pcDNA3.1-hSNORF44-f (ATCC Patent Deposit Designation PTA-100). In a further embodiment, the protein encoded by the nucleic acid analog has a function which is the same as the function of the receptor proteins having the amino acid sequence shown in FIGS. 2A-2B. In another embodiment, the function of the protein encoded by the nucleic acid analog differs from the function of the receptor protein having the amino acid sequence shown in FIGS. 2A-2B. In another embodiment, the variation in the nucleic acid sequence occurs within the transmembrane (TM) region of the protein. In a further embodiment, the variation in the nucleic acid sequence occurs outside of the TM region.

This invention provides the above-described isolated nucleic acid, wherein the nucleic acid is DNA. In an embodiment, the DNA is cDNA. In another embodiment, the DNA is genomic DNA. In still another embodiment, the nucleic acid is RNA. Methods for production and manipulation of nucleic acid molecules are well known in the art.

This invention further provides a nucleic acid which is degenerate with respect to the DNA encoding any of the polypeptides described herein. In an embodiment, the nucleic acid comprises a nucleotide sequence which is degenerate with respect to the nucleotide sequence shown in FIGS. 1A-1C (SEQ ID NO: 1) or the nucleotide sequence contained in the plasmid pCDNA3.1-hSNORF44-f (ATCC Patent Deposit Designation PTA-100), that is, a nucleotide sequence which is translated into the same amino acid sequence.

This invention also encompasses DNAs and cDNAs which encode amino acid sequences which differ from those of the polypeptides of this invention, but which should not produce phenotypic changes.

Alternately, this invention also encompasses DNAs, cDNAs, and RNAs which hybridize to the DNA, cDNA, and RNA of the subject invention. Hybridization methods are well known to those of skill in the art.

The nucleic acids of the subject invention also include nucleic acid molecules coding for polypeptide analogs, fragments or derivatives of antigenic polypeptides which differ from naturally-occurring forms in terms of the identity or location of one or more amino acid residues (deletion analogs containing less than all of the residues specified for the protein, substitution analogs wherein one or more residues specified are replaced by other residues and addition analogs wherein one or more amino acid residues is added to a terminal or medial portion of the polypeptides) and which share some or all properties of naturally-occurring forms. These molecules include: the incorporation of codons “preferred” for expression by selected non-mammalian hosts; the provision of sites for cleavage by restriction endonuclease enzymes; and the provision of additional initial, terminal or intermediate DNA sequences that facilitate construction of readily expressed vectors. The creation of polypeptide analogs is well known to those of skill in the art (Spurney, R. F. et al. (1997); Fong, T. M. et al. (1995); Underwood, D. J. et al. (1994); Graziano, M. P. et al. (1996); Guan X. M. et al. (1995)).

The modified polypeptides of this invention may be transfected into cells either transiently or stably using methods well-known in the art, examples of which are disclosed herein. This invention also provides for binding assays using the modified polypeptides, in which the polypeptide is expressed either transiently or in stable cell lines. This invention further provides a compound identified using a modified polypeptide in a binding assay such as the binding assays described herein.

The nucleic acids described and claimed herein are useful for the information which they provide concerning the amino acid sequence of the polypeptide and as products for the large scale synthesis of the polypeptides by a variety of recombinant techniques. The nucleic acid molecule is useful for generating new cloning and expression vectors, transformed and transfected prokaryotic and eukaryotic host cells, and new and useful methods for cultured growth of such host cells capable of expression of the polypeptide and related products.

This invention also provides an isolated nucleic acid encoding species homologs of the SNORF44 receptor encoded by the nucleic acid sequence shown in FIGS. 1A-1C (SEQ ID NO: 1) or encoded by the plasmid pcDNA3.1-hSNORF44-f (ATCC Patent Deposit Designation PTA-100). In one embodiment, the nucleic acid encodes a mammalian SNORF44 receptor homolog which has substantially the same amino acid sequence as does the SNORF44 receptor encoded by the plasmid pcDNA3.1-hSNORF44-f (ATCC Patent Deposit Designation PTA-100). In another embodiment, the nucleic acid encodes a mammalian SNORF44 receptor homolog which has above 75% amino acid identity to the SNORF44 receptor encoded by the plasmid pcDNA3.1-hSNORF44-f (ATCC Patent Deposit Designation PTA-100); preferably above 85% amino acid identity to the SNORF44 receptor encoded by the plasmid pcDNA3.1-hSNORF44-f (ATCC Patent Deposit Designation PTA-100); most preferably above 95% amino acid identity to the SNORF44 receptor encoded by the plasmid pcDNA3.1-hSNORF44-f (ATCC Patent Deposit Designation PTA-100). In another embodiment, the mammalian SNORF44 receptor homolog has above 70% nucleic acid identity to the SNORF44 receptor gene contained in plasmid pcDNA3.1-hSNORF44-f (ATCC Patent Deposit Designation PTA-100); preferably above 80% nucleic acid identity to the SNORF44 receptor gene contained in the plasmid pcDNA3.1-hSNORF44-f (ATCC Patent Deposit Designation PTA-100); more preferably above 90% nucleic acid identity to the SNORF44 receptor gene contained in the plasmid pcDNA3.1-hSNORF44-f (ATCC Patent Deposit Designation PTA-100). Examples of methods for isolating and purifying species homologs are described elsewhere (e.g., U.S. Pat. No. 5,602,024, WO94/14957, WO97/26853, WO98/15570).

This invention provides an isolated nucleic acid encoding a mammalian SNORF44 receptor. In one embodiment, the nucleic acid is DNA. In another embodiment, the DNA is cDNA. In another embodiment, the DNA is genomic DNA. In another embodiment, the nucleic acid is RNA.

In one embodiment, the mammalian SNORF44 receptor is a human SNORF44 receptor. In another embodiment, the human SNORF44 receptor has an amino acid sequence identical to that encoded by the plasmid pcDNA3.1-hSNORF44-f (ATCC Patent Deposit Designation PTA-100). In another embodiment, the human SNORF44 receptor has an amino acid sequence identical to the amino acid sequence shown in FIGS. 2A-2B (SEQ ID NO: 2).

This invention provides a purified mammalian SNORF44 receptor protein. In one embodiment, the SNORF44 receptor protein is a human SNORF44 receptor protein.

This invention provides a vector comprising the nucleic acid of this invention. This invention further provides a vector adapted for expression in a cell which comprises the regulatory elements necessary for expression of the nucleic acid in the cell operatively linked to the nucleic acid encoding the receptor so as to permit expression thereof, wherein the cell is a bacterial, amphibian, yeast, insect or mammalian cell. In one embodiment, the vector is a baculovirus. In another embodiment, the vector is a plasmid.

This invention provides a plasmid designated pcDNA3.1-hSNORF44-f (ATCC Patent Deposit Designation PTA-100).

This invention further provides for any vector or plasmid which comprises modified untranslated sequences, which are beneficial for expression in desired host cells or for use in binding or functional assays. For example, a vector or plasmid with untranslated sequences of varying lengths may express differing amounts of the polypeptide depending upon the host cell used. In an embodiment, the vector or plasmid comprises the coding sequence of the polypeptide and the regulatory elements necessary for expression in the host cell.

This invention provides for a cell comprising the vector of this invention. In one embodiment, the cell is a non-mammalian cell. In one embodiment, the non-mammalian cell is a Xenopus oocyte cell or a Xenopus melanophore cell. In another embodiment, the cell is a mammalian cell. In another embodiment, the cell is a COS-7 cell, a 293 human embryonic kidney cell, a NIH-3T3 cell, a LM(tk−) cell, a mouse Y1 cell, or a CHO cell. In another embodiment, the cell is an insect cell. In another embodiment, the insect cell is an Sf9 cell, an Sf21 cell or a Trichoplusia ni 5B-4 cell.

This invention provides a membrane preparation isolated from the cell in accordance with this invention.

Furthermore, this invention provides for a nucleic acid probe comprising at least 15 nucleotides, which probe specifically hybridizes with a nucleic acid encoding a mammalian SNORF44 receptor, wherein the probe has a sequence complementary to a unique sequence present within one of the two strands of the nucleic acid encoding the mammalian SNORF44 receptor contained in plasmid pcDNA3.1-hSNORF44-f (ATCC Patent Deposit Designation PTA-100).

This invention further provides a nucleic acid probe comprising at least 15 nucleotides, which probe specifically hybridizes with a nucleic acid encoding a mammalian SNORF44 receptor, wherein the probe has a sequence complementary to a unique sequence present within (a) the nucleic acid sequence shown in FIGS. 1A-1C (SEQ ID NO: 1) or (b) the reverse complement to (a). In one embodiment, the nucleic acid is DNA. In another embodiment, the nucleic acid is RNA.

As used herein, the phrase “specifically hybridizing” means the ability of a nucleic acid molecule to recognize a nucleic acid sequence complementary to its own and to form double-helical segments through hydrogen bonding between complementary base pairs.

The nucleic acids of this invention may be used as probes to obtain homologous nucleic acids from other species and to detect the existence of nucleic acids having complementary sequences in samples.

The nucleic acids may also be used to express the receptors they encode in transfected cells.

The use of a constitutively active receptor encoded by SNORF44 either occurring naturally without further modification or after appropriate point mutations, deletions or the like, allows screening for antagonists and in vivo use of such antagonists to attribute a role to receptor SNORF44 without prior knowledge of the endogenous ligand.

Use of the nucleic acids further enables elucidation of possible receptor diversity and of the existence of multiple subtypes within a family of receptors of which SNORF44 is a member.

Finally, it is contemplated that the receptors of this invention will serve as a valuable tool for designing drugs for treating various pathophysiological conditions such as chronic and acute inflammation, arthritis, autoimmune diseases, transplant rejection, graft vs. host disease, bacterial, fungal, protozoan and viral infections, septicemia, AIDS, pain, psychotic and neurological disorders, including anxiety, depression, schizophrenia, dementia, mental retardation, memory loss, epilepsy, neuromotor disorders, locomotor disorders, respiratory disorders, asthma, eating/body weight disorders including obesity, bulimia, diabetes, anorexia, nausea, hypertension, hypotension, vascular and cardiovascular disorders, ischemia, stroke, cancers, ulcers, urinary retention, sexual/reproductive disorders, circadian rhythm disorders, renal disorders, bone diseases including osteoporosis, benign prostatic hypertrophy, gastrointestinal disorders, nasal congestion, dermatological disorders such as psoriasis, allergies, Parkinson's disease, Alzheimer's disease, acute heart failure, angina disorders, delirium, dyskinesias such as Huntington's disease or Gille's de la Tourette's syndrome, among others and diagnostic assays for such conditions. The receptors of this invention may also serve as a valuable tool for designing drugs for chemoprevention.

Methods of transfecting cells e.g. mammalian cells, with such nucleic acid to obtain cells in which the receptor is expressed on the surface of the cell are well known in the art. (See, for example, U.S. Pat. Nos. 5,053,337; 5,155,218; 5,360,735; 5,472,866; 5,476,782; 5,516,653; 5,545,549; 5,556,753; 5,595,880; 5,602,024; 5,639,652; 5,652,113; 5,661,024; 5,766,879; 5,786,155; and 5,786,157, the disclosures of which are hereby incorporated by reference in their entireties into this application.)

Such transfected cells may also be used to test compounds and screen compound libraries to obtain compounds which bind to the SNORF44 receptor, as well as compounds which activate or inhibit activation of functional responses in such cells, and therefore are likely to do so in vivo. (See, for example, U.S. Pat. Nos. 5,053,337; 5,155,218; 5,360,735; 5,472,866; 5,476,782; 5,516,653; 5,545,549; 5,556,753; 5,595,880; 5,602,024; 5,639,652; 5,652,113; 5,661,024; 5,766,879; 5,786,155; and 5,786,157, the disclosures of which are hereby incorporated by reference in their entireties into this application.)

This invention provides an antibody capable of binding to a mammalian SNORF44 receptor encoded by a nucleic acid encoding a mammalian SNORF44 receptor. In an embodiment of the present invention, the mammalian SNORF44 receptor is a human SNORF44 receptor.

This invention also provides an agent capable of competitively inhibiting the binding of the antibody to a mammalian SNORF44 receptor. In one embodiment, the antibody is a monoclonal antibody or antisera.

Methods of preparing and employing antisense oligonucleotides, antibodies, nucleic acid probes and transgenic animals directed to the SNORF44 receptor are well known in the art. (See, for example, U.S. Pat. Nos. 5,053,337; 5,155,218; 5,360,735; 5,472,866; 5,476,782; 5,516,653; 5,545,549; 5,556,753; 5,595,880; 5,602,024; 5,639,652; 5,652,113; 5,661,024; 5,766,879; 5,786,155; and 5,786,157, the disclosures of which are hereby incorporated by reference in their entireties into this application.)

This invention provides for an antisense oligonucleotide having a sequence capable of specifically hybridizing to RNA encoding a mammalian SNORF44 receptor, so as to prevent translation of such RNA. This invention further provides for an antisense oligonucleotide having a sequence capable of specifically hybridizing to genomic DNA encoding a mammalian SNORF44 receptor, so as to prevent transcription of such genomic DNA. In one embodiment, the oligonucleotide comprises chemically modified nucleotides or nucleotide analogues.

This invention still further provides a pharmaceutical composition comprising (a) an amount of an oligonucleotide in accordance with this invention capable of passing through a cell membrane and effective to reduce expression of a mammalian SNORF44 receptor and (b) a pharmaceutically acceptable carrier capable of passing through the cell membrane.

In one embodiment, the oligonucleotide is coupled to a substance which inactivates mRNA. In another embodiment, the substance which inactivates mRNA is a ribozyme. In another embodiment, the pharmaceutically acceptable carrier comprises a structure which binds to a mammalian SNORF44 receptor on a cell capable of being taken up by the cells after binding to the structure. In another embodiment, the pharmaceutically acceptable carrier is capable of binding to a mammalian SNORF44 receptor which is specific for a selected cell type.

This invention also provides a pharmaceutical composition which comprises an amount of an antibody in accordance with this invention effective to block binding of a ligand to a human SNORF44 receptor and a pharmaceutically acceptable carrier.

This invention further provides a transgenic, nonhuman mammal expressing DNA encoding a mammalian SNORF44 receptor in accordance with this invention. This invention provides a transgenic, nonhuman mammal comprising a homologous recombination knockout of a native mammalian SNORF44 receptor. This invention further provides a transgenic, nonhuman mammal whose genome comprises antisense DNA complementary to DNA encoding a mammalian SNORF44 receptor in accordance with this invention so placed within such genome as to be transcribed into antisense mRNA which is complementary and hybridizes with mRNA encoding the mammalian SNORF44 receptor so as to thereby reduce translation or such mRNA and expression of such receptor. In one embodiment, the DNA encoding the mammalian SNORF44 receptor additionally comprises an inducible promoter. In another embodiment, the DNA encoding the mammalian SNORF44 receptor additionally comprises tissue specific regulatory elements. In another embodiment, the transgenic, nonhuman mammal is a mouse.

This invention provides for a process for identifying a chemical compound which specifically binds to a mammalian SNORF44 receptor which comprises contacting cells containing DNA encoding, and expressing on their cell surface, the mammalian SNORF44 receptor, wherein such cells do not normally express the mammalian SNORF44 receptor, with the compound under conditions suitable for binding, and detecting specific binding of the chemical compound to the mammalian SNORF44 receptor.

This invention further provides for a process for identifying a chemical compound which specifically binds to a mammalian SNORF44 receptor which comprises contacting a membrane preparation from cells containing DNA encoding and expressing on their cell surface the mammalian SNORF44 receptor, wherein such cells do not normally express the mammalian SNORF44 receptor, with the compound under conditions suitable for binding, and detecting specific binding of the chemical compound to the mammalian SNORF44 receptor.

In an embodiment, the mammalian SNORF44 receptor is a human SNORF44 receptor. In another embodiment, the mammalian SNORF44 receptor has substantially the same amino acid sequence as the human SNORF44 receptor encoded by plasmid pcDNA3.1-hSNORF44-f (ATCC Patent Deposit Designation PTA-100).

In another embodiment, the mammalian SNORF44 receptor has substantially the same amino acid sequence as that shown in FIGS. 2A-2B (SEQ ID NO: 2). In another embodiment, the mammalian SNORF44 receptor has the amino acid sequence shown in FIGS. 2A-2B (SEQ ID NO: 2).

In one embodiment, the compound is not previously known to bind to a mammalian SNORF44 receptor. In one embodiment, the cell is an insect cell. In one embodiment, the cell is a mammalian cell. In another embodiment, the cell is nonneuronal in origin. In another embodiment, the nonneuronal cell is a COS-7 cell, 293 human embryonic kidney cell, a CHO cell, a NIH-3T3 cell, a mouse Y1 cell, or a LM(tk−) cell. In another embodiment, the compound is a compound not previously known to bind to a mammalian SNORF44 receptor. This invention provides a compound identified by the preceding processes according to this invention.

This invention still further provides a process involving competitive binding for identifying a chemical compound which specifically binds to a mammalian SNORF44 receptor which comprises separately contacting cells expressing on their cell surface the mammalian SNORF44 receptor, wherein such cells do not normally express the mammalian SNORF44 receptor, with both the chemical compound and a second chemical compound known to bind to the receptor, and with only the second chemical compound, under conditions suitable for binding of such compounds to the receptor, and detecting specific binding of the chemical compound to the mammalian SNORF44 receptor, a decrease in the binding of the second chemical compound to the mammalian SNORF44 receptor in the presence of the chemical compound being tested indicating that such chemical compound binds to the mammalian SNORF44 receptor.

This invention provides a process involving competitive binding for identifying a chemical compound which specifically binds to a mammalian SNORF44 receptor which comprises separately contacting a membrane preparation from cells expressing on their cell surface the mammalian SNORF44 receptor, wherein such cells do not normally express the mammalian SNORF44 receptor, with both the chemical compound and a second chemical compound known to bind to the receptor, and with only the second chemical compound, under conditions suitable for binding of such compounds to the receptor, and detecting specific binding of the chemical compound to the mammalian SNORF44 receptor, a decrease in the binding of the second chemical compound to the mammalian SNORF44 receptor in the presence of the chemical compound being tested indicating that such chemical compound binds to the mammalian SNORF44 receptor.

In an embodiment of the present invention, the second chemical compound is a peptide agonist. Examples of peptide agonists include, but are not limited to, Peptide 1 (SEQ ID NO: 30), Peptide 2 (SEQ ID NO: 31), Peptide 3 (SEQ ID NO: 32), and any peptide comprising His-Phe-Arg.

In an embodiment of the invention, the compound is a peptide which has the following structure: XXXXHFRX-amide (SEQ ID NO: 33), wherein X at positions 1 or 2 is any amino acid present or absent, X at positions 3 or 4 is any amino acid, and X at position 8 is an aromatic amino acid which has a C-terminal amide. Examples of aromatic amino acid are phenylalanine, tyrosine, tryptophan, or histidine.

In one embodiment, the mammalian SNORF44 receptor is a human SNORF44 receptor. In a further embodiment, the cell is an insect cell. In another embodiment, the cell is a mammalian cell. In another embodiment, the cell is nonneuronal in origin. In another embodiment, the nonneuronal cell is a COS-7 cell, 293 human embryonic kidney cell, a CHO cell, a NIH-3T3 cell, a mouse Y1 cell, or a LM(tk−) cell. In another embodiment, the compound is not previously known to bind to a mammalian SNORF44 receptor. This invention provides for a compound identified by the preceding process according to this invention.

This invention provides for a method of screening a plurality of chemical compounds not known to bind to a mammalian SNORF44 receptor to identify a compound which specifically binds to the mammalian SNORF44 receptor, which comprises (a) contacting cells transfected with, and expressing, DNA encoding the mammalian SNORF44 receptor with a compound known to bind specifically to the mammalian SNORF44 receptor; (b) contacting the cells of step (a) with the plurality of compounds not known to bind specifically to the mammalian SNORF44 receptor, under conditions permitting binding of compounds known to bind to the mammalian SNORF44 receptor; (c) determining whether the binding of the compound known to bind to the mammalian SNORF44 receptor is reduced in the presence of the plurality of compounds, relative to the binding of the compound in the absence of the plurality of compounds; and if so (d) separately determining the binding to the mammalian SNORF44 receptor of each compound included in the plurality of compounds, so as to thereby identify any compound included therein which specifically binds to the mammalian SNORF44 receptor.

This invention provides a method of screening a plurality of chemical compounds not known to bind to a mammalian SNORF44 receptor to identify a compound which specifically binds to the mammalian SNORF44 receptor, which comprises (a) contacting a membrane preparation from cells transfected with, and expressing, DNA encoding the mammalian SNORF44 receptor with the plurality of compounds not known to bind specifically to the mammalian SNORF44 receptor under conditions permitting binding of compounds known to bind to the mammalian SNORF44 receptor; (b) determining whether the binding of a compound known to bind to the mammalian SNORF44 receptor is reduced in the presence of the plurality of compounds, relative to the binding of the compound in the absence of the plurality of compounds; and if so (c) separately determining the binding to the mammalian SNORF44 receptor of each compound included in the plurality of compounds, so as to thereby identify any compound included therein which specifically binds to the mammalian SNORF44 receptor.

In one embodiment, the mammalian SNORF44 receptor is a human SNORF44 receptor. In another embodiment, the cell is a mammalian cell. In another embodiment, the mammalian cell is non-neuronal in origin. In a further embodiment, the non-neuronal cell is a COS-7 cell, a 293 human embryonic kidney cell, a LM(tk−) cell, a CHO cell, a mouse Y1 cell, or an NIH-3T3 cell.

This invention also provides a method of detecting expression of a mammalian SNORF44 receptor by detecting the presence of mRNA coding for the mammalian SNORF44 receptor which comprises obtaining total mRNA from the cell and contacting the mRNA so obtained with a nucleic acid probe according to this invention under hybridizing conditions, detecting the presence of mRNA hybridized to the probe, and thereby detecting the expression of the mammalian SNORF44 receptor by the cell.

This invention further provides for a method of detecting the presence of a mammalian SNORF44 receptor on the surface of a cell which comprises contacting the cell with an antibody according to this invention under conditions permitting binding of the antibody to the receptor, detecting the presence of the antibody bound to the cell, and thereby detecting the presence of the mammalian SNORF44 receptor on the surface of the cell.

This invention still further provides a method of determining the physiological effects of varying levels of activity of a mammalian SNORF44 receptor which comprises producing a transgenic, nonhuman mammal in accordance with this invention whose levels of mammalian SNORF44 receptor activity are varied by use of an inducible promoter which regulates mammalian SNORF44 receptor expression.

This invention additionally provides a method of determining the physiological effects of varying levels of activity of a mammalian SNORF44 receptor which comprises producing a panel of transgenic, nonhuman mammals in accordance with this invention each expressing a different amount of a mammalian SNORF44 receptor.

Moreover, this invention provides method for identifying an antagonist capable of alleviating an abnormality wherein the abnormality is alleviated by decreasing the activity of a mammalian SNORF44 receptor comprising administering a compound to a transgenic, nonhuman mammal according to this invention, and determining whether the compound alleviates any physiological and/or behavioral abnormality displayed by the transgenic, nonhuman mammal as a result of overactivity of a mammalian SNORF44 receptor, the alleviation of such an abnormality identifying the compound as an antagonist. In an embodiment, the mammalian SNORF44 receptor is a human SNORF44 receptor.

The invention also provides an antagonist identified by the preceding method according to this invention. This invention further provides a composition, e.g. a pharmaceutical composition comprising an antagonist according to this invention and a carrier, e.g. a pharmaceutically acceptable carrier.

This invention provides a method of treating an abnormality in a subject wherein the abnormality is alleviated by decreasing the activity of a mammalian SNORF44 receptor which comprises administering to the subject an effective amount of the pharmaceutical composition according to this invention so as to thereby treat the abnormality.

In addition, this invention provides a method for identifying an agonist capable of alleviating an abnormality in a subject wherein the abnormality is alleviated by increasing the activity of a mammalian SNORF44 receptor comprising administering a compound to a transgenic, nonhuman mammal according to this invention, and determining whether the compound alleviates any physiological and/or behavioral abnormality displayed by the transgenic, nonhuman mammal, the alleviation of such an abnormality identifying the compound as an agonist. In an embodiment, the mammalian SNORF44 receptor is a human SNORF44 receptor. This invention provides an agonist identified by the preceding method according to this invention. This invention provides a composition, e.g. a pharmaceutical composition comprising an agonist identified by a method according to this invention and a carrier, e.g. a pharmaceutically acceptable carrier.

Moreover, this invention provides a method of treating an abnormality in a subject wherein the abnormality is alleviated by increasing the activity of a mammalian SNORF44 receptor which comprises administering to the subject an effective amount of the pharmaceutical composition of this invention so as to thereby treat the abnormality.

Yet further, this invention provides a method for diagnosing a predisposition to a disorder associated with the activity of a specific mammalian allele which comprises: (a) obtaining DNA of subjects suffering from the disorder; (b) performing a restriction digest of the DNA with a panel of restriction enzymes; (c) electrophoretically separating the resulting DNA fragments on a sizing gel; (d) contacting the resulting gel with a nucleic acid probe capable of specifically hybridizing with a unique sequence included within the sequence of a nucleic acid molecule encoding a mammalian SNORF44 receptor and labeled with a detectable marker; (e) detecting labeled bands which have hybridized to the DNA encoding a mammalian SNORF44 receptor to create a unique band pattern specific to the DNA of subjects suffering from the disorder; (f) repeating steps (a)-(e) with DNA obtained for diagnosis from subjects not yet suffering from the disorder; and (g) comparing the unique band pattern specific to the DNA of subjects suffering from the disorder from step (e) with the band pattern from step (f) for subjects not yet suffering from the disorder so as to determine whether the patterns are the same or different and thereby diagnose predisposition to the disorder if the patterns are the same.

In one embodiment, the disorder is a disorder associated with the activity of a specific mammalian allele is diagnosed.

This invention also provides a method of preparing a purified mammalian SNORF44 receptor according to this invention which comprises: (a) culturing cells which express the mammalian SNORF44 receptor; (b) recovering the mammalian SNORF44 receptor from the cells; and (c) purifying the mammalian SNORF44 receptor so recovered.

This invention further provides a method of preparing a purified mammalian SNORF44 receptor according to this invention which comprises: (a) inserting a nucleic acid encoding the mammalian SNORF44 receptor into a suitable expression vector; (b) introducing the resulting vector into a suitable host cell; (c) placing the resulting host cell in suitable condition permitting the production of the mammalian SNORF44 receptor; (d) recovering the mammalian SNORF44 receptor so produced; and optionally (e) isolating and/or purifying the mammalian SNORF44 receptor so recovered.

Furthermore, this invention provides a process for determining whether a chemical compound is a mammalian SNORF44 receptor agonist which comprises contacting cells transfected with and expressing DNA encoding the mammalian SNORF44 receptor with the compound under conditions permitting the activation of the mammalian SNORF44 receptor, and detecting any increase in mammalian SNORF44 receptor activity, so as to thereby determine whether the compound is a mammalian SNORF44 receptor agonist.

Furthermore, this invention provides a process for determining whether a chemical compound is a mammalian SNORF44 receptor agonist which comprises contacting cells transfected with and expressing DNA encoding the mammalian SNORF44 receptor with the compound under conditions permitting the activation of the mammalian SNORF44 receptor, and detecting any increase in mammalian SNORF44 receptor activity, so as to thereby determine whether the compound is a mammalian SNORF44 receptor agonist.

This invention also provides a process for determining whether a chemical compound is a mammalian SNORF44 receptor antagonist which comprises contacting cells transfected with and expressing DNA encoding the mammalian SNORF44 receptor with the compound in the presence of a known mammalian SNORF44 receptor agonist, under conditions permitting the activation of the mammalian SNORF44 receptor, and detecting any decrease in mammalian SNORF44 receptor activity, so as to thereby determine whether the compound is a mammalian SNORF44 receptor antagonist.

This invention also provides a process for determining whether a chemical compound is a mammalian SNORF44 receptor antagonist which comprises contacting cells transfected with and expressing DNA encoding the mammalian SNORF44 receptor with the compound in the presence of a known mammalian SNORF44 receptor agonist, under conditions permitting the activation of the mammalian SNORF44 receptor, and detecting any decrease in mammalian SNORF44 receptor activity, so as to thereby determine whether the compound is a mammalian SNORF44 receptor antagonist.

In an embodiment, the mammalian SNORF44 receptor is a human SNORF44 receptor.

This invention still further provides a composition, for example a pharmaceutical composition, which comprises an amount of a mammalian SNORF44 receptor agonist determined by a process according to this invention effective to increase activity of a mammalian SNORF44 receptor and a carrier, for example, a pharmaceutically acceptable carrier. In one embodiment, the mammalian SNORF44 receptor agonist is not previously known.

Also, this invention provides a composition, for example a pharmaceutical composition, which comprises an amount of a mammalian SNORF44 receptor antagonist determined by a process according to this invention effective to reduce activity of a mammalian SNORF44 receptor and a carrier, for example, a pharmaceutically acceptable carrier. Also, this invention provides a composition, for example a pharmaceutical composition, which comprises an amount of a mammalian SNORF44 receptor antagonist determined by a process according to this invention effective to reduce activity of a mammalian SNORF44 receptor and a carrier, for example, a pharmaceutically acceptable carrier.

In one embodiment, the mammalian SNORF44 receptor antagonist is not previously known. In an embodiment, the mammalian SNORF44 receptor antagonist is a human SNORF44 receptor antagonist.

This invention moreover provides a process for determining whether a chemical compound specifically binds to and activates a mammalian SNORF44 receptor, which comprises contacting cells producing a second messenger response and expressing on their cell surface the mammalian SNORF44 receptor, wherein such cells do not normally express the mammalian SNORF44 receptor, with the chemical compound under conditions suitable for activation of the mammalian SNORF44 receptor, and measuring the second messenger response in the presence and in the absence of the chemical compound, a change, e.g. an increase, in the second messenger response in the presence of the chemical compound indicating that the compound activates the mammalian SNORF44 receptor.

In one embodiment, the second messenger response comprises chloride channel activation and the change in second messenger is an increase in the level of chloride current. In another embodiment, the second messenger response comprises change in intracellular calcium levels and the change in second messenger is an increase in the measure of intracellular calcium. In another embodiment, the second messenger response comprises release of inositol phosphate and the change in second messenger is an increase in the level of inositol phosphate. In another embodiment, the second messenger response comprises release of arachidonic acid and the change in second messenger is an increase in the level of arachidonic acid. In yet another embodiment, the second messenger response comprises GTPγS ligand binding and the change in second messenger is an increase in GTPγS ligand binding. In another embodiment, the second messenger response comprises activation of MAP kinase and the change in second messenger response is an increase in MAP kinase activation. In a further embodiment, the second messenger response comprises cAMP accumulation and the change in second messenger response is a reduction in cAMP accumulation.

This invention still further provides a process for determining whether a chemical compound specifically binds to and inhibits activation of a mammalian SNORF44 receptor, which comprises separately contacting cells producing a second messenger response and expressing on their cell surface the mammalian SNORF44 receptor, wherein such cells do not normally express the mammalian SNORF44 receptor, with both the chemical compound and a second chemical compound known to activate the mammalian SNORF44 receptor, and with only the second chemical compound, under conditions suitable for activation of the mammalian SNORF44 receptor, and measuring the second messenger response in the presence of only the second chemical compound and in the presence of both the second chemical compound and the chemical compound, a smaller change, e.g. increase, in the second messenger response in the presence of both the chemical compound and the second chemical compound than in the presence of only the second chemical compound indicating that the chemical compound inhibits activation of the mammalian SNORF44 receptor.

In an embodiment of the present invention, the second chemical compound is a peptide agonist. Examples of peptide agonists include, but are not limited to, Peptide 1 (SEQ ID NO: 30), Peptide 2 (SEQ ID NO: 31), and Peptide 3 (SEQ ID NO: 32), and any peptide comprising His-Phe-Arg.

In an embodiment of the invention, the compound is a peptide which has the following structure: XXXXHFRX-amide (SEQ ID NO: 33), wherein X at positions 1 or 2 is any amino acid present or absent, X at positions 3 or 4 is any amino acid, and X at position 8 is an aromatic amino acid which has a C-terminal amide. Examples of aromatic amino acid are phenylalanine, tyrosine, tryptophan, or histidine.

In one embodiment, the second messenger response comprises chloride channel activation and the change in second messenger response is a smaller increase in the level of chloride current in the presence of both the chemical compound and the second chemical compound than in the presence of only the second chemical compound. In another embodiment, the second messenger response comprises change in intracellular calcium levels and the change in second messenger response is a smaller increase in the measure of intracellular calcium in the presence of both the chemical compound and the second chemical compound than in the presence of only the second chemical compound. In another embodiment, the second messenger response comprises release of inositol phosphate and the change in second messenger response is a smaller increase in the level of inositol phosphate in the presence of both the chemical compound and the second chemical compound than in the presence of only the second chemical compound.

In one embodiment, the second messenger response comprises activation of MAP kinase and the change in second messenger response is a smaller increase in the level of MAP kinase activation in the presence of both the chemical compound and the second chemical compound than in the presence of only the second chemical compound. In another embodiment, the second messenger response comprises change in cAMP levels and the change in second messenger response is a smaller change in the level of cAMP in the presence of both the chemical compound and the second chemical compound than in the presence of only the second chemical compound. In another embodiment, the second messenger response comprises release of arachidonic acid and the change in second messenger response is an increase in the level of arachidonic acid levels in the presence of both the chemical compound and the second chemical compound than in the presence of only the second chemical compound. In a further embodiment, the second messenger response comprises GTPγS ligand binding and the change in second messenger is a smaller increase in GTPγS ligand binding in the presence of both the chemical compound and the second chemical compound than in the presence of only the second chemical compound.

In one embodiment, the mammalian SNORF44 receptor is a human SNORF44 receptor. In another embodiment, the cell is an insect cell. In another embodiment, the cell is a mammalian cell. In another embodiment, the mammalian cell is nonneuronal in origin. In another embodiment, the nonneuronal cell is a COS-7 cell, CHO cell, 293 human embryonic kidney cell, NIH-3T3 cell or LM(tk−) cell. In another embodiment, the compound is not previously known to bind to a mammalian SNORF44 receptor.

Further, this invention provides a compound determined by a process according to this invention and a composition, for example, a pharmaceutical composition, which comprises an amount of a mammalian SNORF44 receptor agonist determined to be such by a process according to this invention effective to increase activity of a mammalian SNORF44 receptor and a carrier, for example, a pharmaceutically acceptable carrier. In one embodiment, the mammalian SNORF44 receptor agonist is not previously known.

This invention also provides a composition, for example, a pharmaceutical composition, which comprises an amount of a mammalian SNORF44 antagonist determined to be such by a process according to this invention, effective to reduce activity of the mammalian SNORF44 receptor and a carrier, for example a pharmaceutically acceptable carrier. In one embodiment, the mammalian SNORF44 antagonist is not previously known.

This invention yet further provides a method of screening a plurality of chemical compounds not known to activate a mammalian SNORF44 receptor to identify a compound which activates the mammalian SNORF44 receptor which comprises: (a) contacting cells transfected with and expressing the mammalian SNORF44 receptor with the plurality of compounds not known to activate the mammalian SNORF44 receptor, under conditions permitting activation of the mammalian SNORF44 receptor; (b) determining whether the activity of the mammalian SNORF44 receptor is increased in the presence of one or more of the compounds; and if so (c) separately determining whether the activation of the mammalian SNORF44 receptor is increased by any compound included in the plurality of compounds, so as to thereby identify each compound which activates the mammalian SNORF44 receptor.

In an embodiment, the mammalian SNORF44 receptor is a human SNORF44 receptor.

This invention provides a method of screening a plurality of chemical compounds not known to inhibit the activation of a mammalian SNORF44 receptor to identify a compound which inhibits the activation of the mammalian SNORF44 receptor, which comprises: (a) contacting cells transfected with and expressing the mammalian SNORF44 receptor with the plurality of compounds in the presence of a known mammalian SNORF44 receptor agonist, under conditions permitting activation of the mammalian SNORF44 receptor; (b) determining whether the extent or amount of activation of the mammalian SNORF44 receptor is reduced in the presence of one or more of the compounds, relative to the extent or amount of activation of the mammalian SNORF44 receptor in the absence of such one or more compounds; and if so (c) separately determining whether each such compound inhibits activation of the mammalian SNORF44 receptor for each compound included in the plurality of compounds, so as to thereby identify any compound included in such plurality of compounds which inhibits the activation of the mammalian SNORF44 receptor.

In an embodiment of the invention, the compound is a peptide which has the following structure: XXXXHFRX-amide (SEQ ID NO: 33), wherein X at positions 1 or 2 is any amino acid present or absent, X at positions 3 or 4 is any amino acid, and X at position 8 is an aromatic amino acid which has a C-terminal amide. Examples of aromatic amino acid are phenylalanine, tyrosine, tryptophan, or histidine.

In one embodiment, the mammalian SNORF44 receptor is a human SNORF44 receptor. In another embodiment, wherein the cell is a mammalian cell. In another embodiment, the mammalian cell is non-neuronal in origin. In another embodiment, the non-neuronal cell is a COS-7 cell, a 293 human embryonic kidney cell, a LM(tk−) cell or an NIH-3T3 cell.

This invention also provides a composition, for example, a pharmaceutical composition, comprising a compound identified by a method according to this invention in an amount effective to increase mammalian SNORF44 receptor activity and a carrier, for example, a pharmaceutically acceptable carrier.

This invention still further provides a composition, for example, a pharmaceutical composition, comprising a compound identified by a method according to this invention in an amount effective to decrease mammalian SNORF44 receptor activity and a carrier, for example, a pharmaceutically acceptable carrier.

Furthermore, this invention provides a method of treating an abnormality in a subject wherein the abnormality is alleviated by increasing the activity of a mammalian SNORF44 receptor which comprises administering to the subject a compound which is a mammalian SNORF44 receptor agonist in an amount effective to treat the abnormality.

In one embodiment, the abnormality is a regulation of a steroid hormone disorder, an epinephrine release disorder, a gastrointestinal disorder, a cardiovascular disorder, an electrolyte balance disorder, hypertension, diabetes, a respiratory disorder, asthma, a reproductive function disorder, an immune disorder, an endocrine disorder, a musculoskeletal disorder, a neuroendocrine disorder, a cognitive disorder, a memory disorder, somatosensory and neurotransmission disorders, metabolic disorders, a motor coordination disorder, a sensory integration disorder, a motor integration disorder, a dopaminergic function disorder, an appetite disorder, such as anorexia or obesity, a sensory transmission disorder, drug addiction, an olfaction disorder, an autonomic nervous system disorder, pain, neuropsychiatric disorders, affective disorder, migraine, circadian disorders, visual disorders, urinary disorders, blood coagulation-related disorders, developmental disorders, or ischemia-reperfusion injury-related diseases.

This invention additionally provides a method of treating an abnormality in a subject wherein the abnormality is alleviated by decreasing the activity of a mammalian SNORF44 receptor which comprises administering to the subject a compound which is a mammalian SNORF44 receptor antagonist in an amount effective to treat the abnormality.

In one embodiment, the abnormality is a regulation of a steroid hormone disorder, an epinephrine release disorder, a gastrointestinal disorder, a cardiovascular disorder, an electrolyte balance disorder, hypertension, diabetes, a respiratory disorder, asthma, a reproductive function disorder, an immune disorder, an endocrine disorder, a musculoskeletal disorder, a neuroendocrine disorder, a cognitive disorder, a memory disorder, somatosensory and neurotransmission disorders, metabolic disorders, a motor coordination disorder, a sensory integration disorder, a motor integration disorder, a dopaminergic function disorder, an appetite disorder, such as anorexia or obesity, a sensory transmission disorder, drug addiction, an olfaction disorder, an autonomic nervous system disorder, pain, neuropsychiatric disorders, affective disorder, migraine, circadian disorders, visual disorders, urinary disorders, blood coagulation-related disorders, developmental disorders, or ischemia-reperfusion injury-related diseases.

In an embodiment of the invention, the compound is a peptide which has the following structure: XXXXHFRX-amide (SEQ ID NO: 33), wherein X at positions 1 or 2 is any amino acid present or absent, X at positions 3 or 4 is any amino acid, and X at position 8 is an aromatic amino acid which has a C-terminal amide. Examples of aromatic amino acid are phenylalanine, tyrosine, tryptophan, or histidine.

In one embodiment, the mammalian SNORF44 receptor is a human SNORF44 receptor.

This invention also provides a process for making a composition of matter which specifically binds to a mammalian SNORF44 receptor which comprises identifying a chemical compound using a process in accordance with this invention and then synthesizing the chemical compound or a novel structural and functional analog or homolog thereof.

This invention further provides a process for preparing a composition, for example a pharmaceutical composition which comprises admixing a carrier, for example, a pharmaceutically acceptable carrier, and a pharmaceutically effective amount of a chemical compound identified by a process in accordance with this invention or a novel structural and functional analog or homolog thereof.

This invention further provides a process for preparing a composition, for example a pharmaceutical composition which comprises identifying a chemical compound by a process in accordance with this invention or a novel structural and functional analog or homolog thereof, recovering the chemical compound free of any receptor, and then admixing a carrier, for example, a pharmaceutically acceptable carrier, and a pharmaceutically effective amount of the chemical compound.

In one embodiment, the mammalian SNORF44 receptor is a human SNORF44 receptor.

Thus, once the gene for a targeted receptor subtype is cloned, it is placed into a recipient cell which then expresses the targeted receptor subtype on its surface. This cell, which expresses a single population of the targeted human receptor subtype, is then propagated resulting in the establishment of a cell line. This cell line, which constitutes a drug discovery system, is used in two different types of assays: binding assays and functional assays. In binding assays, the affinity of a compound for both the receptor subtype that is the target of a particular drug discovery program and other receptor subtypes that could be associated with side effects are measured. These measurements enable one to predict the potency of a compound, as well as the degree of selectivity that the compound has for the targeted receptor subtype over other receptor subtypes. The data obtained from binding assays also enable chemists to design compounds toward or away from one or more of the relevant subtypes, as appropriate, for optimal therapeutic efficacy. In functional assays, the nature of the response of the receptor subtype to the compound is determined. Data from the functional assays show whether the compound is acting to inhibit or enhance the activity of the receptor subtype, thus enabling pharmacologists to evaluate compounds rapidly at their ultimate human receptor subtypes targets permitting chemists to rationally design drugs that will be more effective and have fewer or substantially less severe side effects than existing drugs.

Approaches to designing and synthesizing receptor subtype-selective compounds are well known and include traditional medicinal chemistry and the newer technology of combinatorial chemistry, both of which are supported by computer-assisted molecular modeling. With such approaches, chemists and pharmacologists use their knowledge of the structures of the targeted receptor subtype and compounds determined to bind and/or activate or inhibit activation of the receptor subtype to design and synthesize structures that will have activity at these receptor subtypes.

Combinatorial chemistry involves automated synthesis of a variety of novel compounds by assembling them using different combinations of chemical building blocks. The use of combinatorial chemistry greatly accelerates the process of generating compounds. The resulting arrays of compounds are called libraries and are used to screen for compounds (“lead compounds”) that demonstrate a sufficient level of activity at receptors of interest. Using combinatorial chemistry it is possible to synthesize “focused” libraries of compounds anticipated to be highly biased toward the receptor target of interest.

Once lead compounds are identified, whether through the use of combinatorial chemistry or traditional medicinal chemistry or otherwise, a variety of homologs and analogs are prepared to facilitate an understanding of the relationship between chemical structure and biological or functional activity. These studies define structure activity relationships which are then used to design drugs with improved potency, selectivity and pharmacokinetic properties. Combinatorial chemistry is also used to rapidly generate a variety of structures for lead optimization. Traditional medicinal chemistry, which involves the synthesis of compounds one at a time, is also used for further refinement and to generate compounds not accessible by automated techniques. Once such drugs are defined the production is scaled up using standard chemical manufacturing methodologies utilized throughout the pharmaceutical and chemistry industry.

This invention will be better understood from the Experimental Details which follow. However, one skilled in the art will readily appreciate that the specific methods and results discussed are merely illustrative of the invention as described more fully in the claims which follow thereafter.

### EXPERIMENTAL DETAILS

Materials and Methods

Isolation of a Fragment of the Human SNORF44 Receptor by MOPAC (Mixed Oligonucleotide Primed Amplification of cDNA)

Approximately 10 ng human hypothalamic cDNA (Clontech, Palo Alto, Calif.) was used for degenerate MOPAC PCR using Taq DNA polymerase (Roche, Indianapolis, Ind.) and the following degenerate oligonucleotides: JAB401, designed based on an alignment of the sixth transmembrane domain of more than 180 members of the rhodopsin superfamily of G protein coupled receptors; and JAB404, designed based on an alignment of the seventh transmembrane domain of the same rhodopsin superfamily.

The conditions for the MOPAC PCR reaction were as follows: 3 minute hold at 94° C.; 10 cycles of 1 minute at 94° C., 1 minute 45 seconds at 44° C., 2 minutes at 72° C.; 30 cycles of 94° C. for 1 minute, 49° C. for 1 minute 45 seconds, 2 minutes at 72° C.; 4 minute hold at 72° C.; 4° C. hold until ready for agarose gel electrophoresis.

The products were run on a 1% agarose TAE gel and bands of the expected size (˜150 bp) were cut from the gel, purified using the QIAQUICK gel extraction kit (QIAGEN, Chatsworth, Calif.), and subcloned into the TA cloning vector (Invitrogen, San Diego, Calif.). White (insert-containing) colonies were picked and subjected to PCR using pCR2.1 vector primers JAB1 and JAB2 using the Expand Long Template PCR System and the following protocol: 94° C. hold for 3 minutes; 35 cycles of 94° C. for 1 minute, 68° C. for 1 minute 15 seconds; 2 minute hold at 68° C., 4° C. hold until the products were ready for purification. PCR products were purified by isopropanol precipitation (10 ul PCR product, 18 ul low TE, 10.5 ul 2M NaClO₄, and 21.5 ul isopropanol) and sequenced using the ABI Big Dye cycle sequencing protocol and ABI 377 sequencers (ABI, Foster City, Calif.). Nucleotide and amino acid sequence analyses were performed using the Wisconsin Package (GCG, Genetics Computer Group, Madison, Wis.). One PCR product (MPR133-HUMHT-091) was determined to be a novel G protein-coupled receptor-like sequence fragment, between the sixth and seventh transmembrane domains, based on database searches and its homology to other known G protein-coupled receptors. This novel sequence was designated hSNORF44. Through BLAST and GRAIL analysis, the DNA sequence corresponding to the region of the SNORF44 gene coding for the third transmembrane domain through a stop codon following the seventh transmembrane domain was found to be represented in a large human genomic DNA entry in GenEmbl (GenBank accession number AC005961). This entry consisted of eight unordered pieces of human genomic DNA from chromosome 11q12.2. The partial SNORF44 sequence represented in AC005961 was interrupted by four introns, so that the coding sequence for the region of the gene encoding the third through the seventh transmembrane domains spanned more than 10,000 bp. AC005961 was not annotated as containing a GPCR-encoding gene.

Isolation of the Full-Length Human SNORF44 Receptor

The sequence corresponding to the third transmembrane domain through a putative stop codon after the seventh transmembrane domain was inferred through sequence analysis of AC005961 and confirmed by PCR of human spinal cord cDNA with primers specific to the third transmembrane domain (JAB433) and the proposed 3′untranslated region (JAB434). To determine the 5′ end of the coding sequence of human SNORF44 (from the initiating methionine to the third transmembrane domain), we utilized Clontech's Marathon

Ready human fetal brain cDNA for 5′ Rapid Amplification of cDNA ends (RACE). 5 ul of template was subjected to PCR with JAB440 and AP1 for the first round of PCR, after which 1 ul of product from the first PCR reaction was used as a template for a second round of PCR using JAB441 and AP2 as primers. The PCR protocol for both of these PCR reactions was as follows: 1 minute at 94° C.; 5 cycles of 94° C. for 30 seconds and 72° C. for 4 minutes; 5 cycles of 94° C. for 30 seconds and 70° C. for 4 minutes; 30 cycles of 94° C. for 30 seconds and 68° C. for 4 minutes; 68° C. hold for 5 minutes, and 4° C. hold until the products were ready for analysis. A 700 bp PCR product was agarose-gel purified as described above, and directly sequenced with JAB442. Sequence was also confirmed by subcloning the fragments into pCR2.1, and sequencing the subcloned fragments with a T7 primer as described above. Sequences were analyzed using the Wisconsin Package (GCG, Genetics Computer Group, Madison, Wis.). This product contained sequence information for SNORF44 from the 5′ untranslated region upstream from a potential initiating methionine to the coding sequence of the third transmembrane domain.

After determining the full-length coding sequence of SNORF44, the entire coding region was amplified in five independent PCR reactions from human spinal cord cDNA using the Expand Long Template PCR System and the following primers: JAB447, a forward primer 5′ to the beginning of the coding sequence with a BamHI site incorporated into the 5′ end, and JAB448, a reverse primer 3′ to the stop codon with a HindIII site incorporated into the 5′ end. The products from these reactions were gel purified from a 1% agarose TAE gel and ligated into pCR2.1. One clone from each reaction was chosen and sequenced with the SNORF44-specific primers JAB455, JAB452, JAB433, JAB439, JAB435, JAB432, JAB438, JAB440, and JAB446, as well as the pCR2.1 vector primers T7 and M13R. The double-stranded contiguous sequence from each clone was assembled using the Wisconsin Package (GCG, Genetics Computer Group, Madison, Wis.) and Sequencher 3.0 (Gene Codes Corporation, Ann Arbor, Mich.). Sequence analysis of the consensus of the sequences of the five clones confirmed the actual sequence of the full-length human SNORF44 receptor. At this point the SNORF44 inserts were cut from the pCR2.1 vector and subcloned into pcDNA3.1(−). Since there was not a single clone representing the consensus sequence of SNORF44 from beginning to end, the 5′ end of one of the SNORF44 clones (in pcDNA3.1) was cut with BamHI and BbrI, and ligated into the same site of another construct to generate a SNORF44 receptor sequence representing the consensus sequence of the five clones in pcDNA3.1(−). This construct was given the name BN16.

Oligonucleotide Primers

The following is a list of primers and their associated sequences which were used in the cloning and sequencing of this receptor:

| | | |
|---|---|---|
| AP1: | 5′-CCATCCTAATACGACTCACTATAGGGC-3′ | (SEQ ID NO: 3) |
| AP2: | 5′-ACTCACTATAGGGCTCGAGCGGC-3′ | (SEQ ID NO: 4) |
| JAB1: | 5′-TTATGCTTCCGGCTCGTATGTTGTG-3′ | (SEQ ID NO: 5) |
| JAB2: | 5′-ATGTGCTGCAAGGCGATTAAGTTGGG-3′ | (SEQ ID NO: 6) |
| JAB401: | 5′-GTNGBNITITTYIYIITIWGYTGG-3′ | (SEQ ID NO: 7) |
| JAB404: | 5′-NARRAAIGCRTAIADIAIIGGRTT-3′ | (SEQ ID NO: 8) |
| JAB432: | 5′-TTCATTCATIAAATGCATAGACAATGGG-3′ | (SEQ ID NO: 9) |
| JAB433: | 5′-CAGGTGCTTTCATTTGCAAGATGGTG-3′ | (SEQ ID NO: 10) |
| JAB434: | 5′-CAATCTGAAGGGCATTAATTATGAAG-3′ | (SEQ ID NO: 11) |
| JAB435: | 5′-CACCATTCCATGTTGTCCATATGATG-3′ | (SEQ ID NO: 12) |
| JAB438: | 5′-GTACAGAATAAGCATCACCATAAGAGG-3′ | (SEQ ID NO: 13) |
| JAB439: | 5′-CTCTTATGGTGATGCTTATTCTGTACAG-3′ | (SEQ ID NO: 14) |
| JAB440: | 5′-TGCACAAGTCCCTGGTGCCTTTCCAC-3′ | (SEQ ID NO: 15) |
| JAB441: | 5′-GCAATGCAGGTCATAGTGAGGATTTC-3′ | (SEQ ID NO: 16) |
| JAB442: | 5′-CACAACAGCGGTAGACTGGACAAATGG-3′ | (SEQ ID NO: 17) |
| JAB444: | 5′-GGAGCAGATAAAGATGTTGGTGACGG-3′ | (SEQ ID NO: 18) |
| JAB445: | 5′-CCACGTAGAACACCAGAGCATTGCC-3′ | (SEQ ID NO: 19) |
| JAB446: | 5′-CCGGTGAGCACGAGGGCCAGCTTGG-3′ | (SEQ ID NO: 20) |
| JAB447: | 5′-ATCTATGGATCCTATCGTGGGTTTGATCCCTGAGCTG-3′ | (SEQ ID NO: 21) |
| JAB448: | 5′-ATCTATAAGCTTTTCTCTTTGGGTTACAATCTGAAGGG-3′ | (SEQ ID NO: 22) |
| JAB452: | 5′-TCACCAACATCTTTATCTGCTCCTTGG-3′ | (SEQ ID NO: 23) |
| JAB455: | 5′-CAGCAATGCAGGCGCTTAACATTACCC-3′ | (SEQ ID NO: 24) |
| T7: | 5′-TAATACGACTCACTATAGGG-3′ | (SEQ ID NO: 25) |
| M13R: | 5′-CAGGAAACAGCTATGA-3′ | (SEQ ID NO: 26) |

Isolation of Species Homologs of SNORF44 Receptor

A nucleic acid sequence encoding a SNORF44 receptor from other species may be isolated using standard molecular biology techniques and approaches such as those described below:

Approach #1: A genomic library (e.g., cosmid, phage, P1, BAC, YAC) generated from the species of interest may be screened with a ³²P-labeled oligonucleotide probe corresponding to a fragment of the human SNORF44 receptor whose sequence is shown in FIGS. 1A-1C to isolate a genomic clone. The full-length sequence may be obtained by sequencing this genomic clone. If one or more introns are present in the gene, the full-length intronless gene may be obtained from cDNA using standard molecular biology techniques. For example, a forward PCR primer designed in the 5′UT and a reverse PCR primer designed in the 3′UT may be used to amplify a full-length, intronless receptor from cDNA. Standard molecular biology techniques could be used to subclone this gene into a mammalian expression vector.

Approach #2: Standard molecular biology techniques may be used to screen commercial cDNA phage libraries of the species of interest by hybridization under reduced stringency with a ³²P-labeled oligonucleotide probe corresponding to a fragment of the sequences shown in FIGS. 1A-1C. One may isolate a full-length SNORF44 receptor by obtaining a plaque purified clone from the lambda libraries and then subjecting the clone to direct DNA sequencing. Alternatively, standard molecular biology techniques could be used to screen cDNA plasmid libraries by PCR amplification of library pools using primers designed against a partial species homolog sequence. A full-length clone may be isolated by Southern hybridization of colony lifts of positive pools with a ³²P-oligonucleotide probe.

Approach #3: 3′ and 5′ RACE may be utilized to generate PCR products from cDNA derived from the species of interest expressing SNORF44 which contain the additional sequence of SNORF44. These RACE PCR products may then be sequenced to determine the additional sequence. This new sequence is then used to design a forward PCR primer in the 5′UT and a reverse primer in the 3′UT. These primers are then used to amplify a full-length SNORF44 clone from cDNA.

Examples of other species include, but are not limited to, rat, mouse, dog, monkey, hamster and guinea pig.

Host Cells

A broad variety of host cells can be used to study heterologously expressed proteins. These cells include but are not limited to mammalian cell lines such as; COS-7, CHO, LM (tk⁻), HEK293, etc.; insect cell lines such as; Sf9, Sf21, Trichoplusia ni 5B-4, etc.; amphibian cells such as Xenopus oocytes; assorted yeast strains; assorted bacterial cell strains; and others. Culture conditions for each of these cell types is specific and is known to those familiar with the art.

COS-7 cells are grown on 150 mm plates in DMEM with supplements (Dulbecco's Modified Eagle Medium with 10% bovine calf serum, 4 mM glutamine, 100 units/ml penicillin 100 μg/ml streptomycin) at 37° C., 5% CO₂. Stock plates of COS-7 cells are trypsinized and split 1:6 every 3-4 days.

Transient Expression

DNA encoding proteins to be studied can be transiently expressed in a variety of mammalian, insect, amphibian, yeast, bacterial and other cells lines by several transfection methods including but not limited to; calcium phosphate-mediated, DEAE-dextran mediated; liposomal-mediated, viral-mediated, electroporation-mediated, and microinjection delivery. Each of these methods may require optimization of assorted experimental parameters depending on the DNA, cell line, and the type of assay to be subsequently employed.

A typical protocol for the DEAE-dextran method as applied to COS-7 and HEK293 cells is described as follows. Cells to be used for transfection are split 24 hours prior to the transfection to provide flasks which are 70-80% confluent at the time of transfection. Briefly, 8 μg of receptor DNA plus 8 μg of any additional DNA needed (e.g. G_{α} protein expression vector, reporter construct, antibiotic resistance marker, mock vector, etc.) are added to 9 ml of complete DMEM plus DEAE-dextran mixture (10 mg/ml in PBS). Cells plated into a T225 flask (sub-confluent) are washed once with PBS and the DNA mixture is added to each flask. The cells are allowed to incubate for 30 minutes at 37° C., 5% CO₂. Following the incubation, 36 ml of complete DMEM with 80 μM chloroquine is added to each flask and allowed to incubate an additional 3 hours. The medium is then aspirated and 24 ml of complete medium containing 10% DMSO for exactly 2 minutes and then aspirated. The cells are then washed 2 times with PBS and 30 ml of complete DMEM added to each flask. The cells are then allowed to incubate over night. The next day the cells are harvested by trypsinization and reseeded into 96 well plates.

Stable Expression

Heterologous DNA can be stably incorporated into host cells, causing the cell to perpetually express a foreign protein. Methods for the delivery of the DNA into the cell are similar to those described above for transient expression but require the co-transfection of an ancillary gene to confer drug resistance on the targeted host cell. The ensuing drug resistance can be exploited to select and maintain cells that have taken up the DNA. An assortment of resistance genes are available including but not restricted to neomycin, kanamycin, and hygromycin. For purposes of studies concerning the receptor of this invention, stable expression of a heterologous receptor protein is typically carrier out in, mammalian cells including but not necessarily restricted to, CHO, HEK293, LM(tk−), etc. In addition native cell lines that naturally carry and express the nucleic acid sequences for the receptor may be used without the need to engineer the receptor complement.

Functional Assays

Cells expressing the receptor DNA of this invention may be used to screen for ligands to said receptor using functional assays. Once a ligand is identified the same assays may be used to identify agonists or antagonists of the receptor that may be employed for a variety of therapeutic purposes. It is well known to those in the art that the over-expression of a G-protein coupled receptor can result in the constitutive activation of intracellular signaling pathways. In the same manner, over-expression of the receptors of the present invention in any cell line as described above, can result in the activation of the functional responses described below, and any of the assays herein described can be used to screen for agonist, partial agonist, inverse agonist and antagonist ligands of the SNORF44 receptor.

A wide spectrum of assays can be employed to screen for the presence of SNORF44 receptor ligands. These assays range from traditional measurements of total inositol phosphate accumulation, cAMP levels, intracellular calcium mobilization, and potassium currents, for example; to systems measuring these same second messengers but which have been modified or adapted to be of higher throughput, more generic and more sensitive; to cell based assays reporting more general cellular events resulting from receptor activation such as metabolic changes, differentiation, cell division/proliferation. Description of several such assays follow.

Cyclic AMP (cAMP) Assay

The receptor-mediated stimulation or inhibition of cyclic AMP (cAMP) formation may be assayed in cells expressing the receptors. COS-7 cells are transiently transfected with the receptor gene using the DEAE-dextran method and plated in 96-well plates. 48 hours after transfection, cells are washed twice with Dulbecco's phosphate buffered saline (PBS) supplemented with 10 mM HEPES, 10 mM glucose and 5 mM theophylline and are incubated in the same buffer for 20 min at 37° C., in 5% CO₂. Test compounds are added and cells are incubated for an additional 10 min at 37° C. The medium is then aspirated and the reaction stopped by the addition of 100 mM HCl. The plates are stored at −20° C. for 2-5 days. For cAMP measurement, plates are thawed and the cAMP content in each well is measured by cAMP Scintillation Proximity Assay (Amersham Pharmacia Biotech). Radioactivity is quantified using microbeta Trilux counter (Wallac).

Arachidonic Acid Release Assay

Cells expressing the receptor are seeded into 96 well plates or other vessels and grown for 3 days in medium with supplements. ³H-arachidonic acid (specific activity=0.75 μCi/ml) is delivered as a 100 μL aliquot to each well and samples are incubated at 37° C., 5% CO₂ for 18 hours. The labeled cells are washed three times with medium. The wells are then filled with medium and the assay is initiated with the addition of test compounds or buffer in a total volume of 250 μL. Cells are incubated for 30 min at 37° C., 5% CO₂. Supernatants are transferred to a microtiter plate and evaporated to dryness at 75° C. in a vacuum oven. Samples are then dissolved and resuspended in 25 μL distilled water. Scintillant (300 μL) is added to each well and samples are counted for ³H in a Trilux plate reader. Data are analyzed using nonlinear regression and statistical techniques available in the GraphPAD Prism package (San Diego, Calif.).

Intracellular Calcium Mobilization Assays

The intracellular free calcium (Ca²⁺) concentration may be measured by microspectrofluorimetry using the fluorescent indicator dye Fura-2/AM (Bush et al., 1991). Cells expressing the receptor are seeded onto a 35 mm culture dish containing a glass coverslip insert and allowed to adhere overnight. Cells are then washed with HBS and loaded with 100 μL of Fura-2/AM (10 μM) for 20 to 40 min. After washing with HBS to remove the Fura-2/AM solution, cells are equilibrated in HBS for 10 to 20 min. Cells are then visualized under the 40× objective of a Leitz Fluovert FS microscope and fluorescence emission is determined at 510 nM with excitation wavelengths alternating between 340 nM and 380 nM. Raw fluorescence data are converted to Ca²⁺ concentrations using standard Ca²⁺ concentration curves and software analysis techniques.

In another method, the measurement of intracellular Ca²⁺ can also be performed on a 96-well (or higher) format and with alternative Ca²⁺-sensitive indicators, preferred examples of these are: aequorin, Fluo-3, Fluo-4, Fluo-5, Calcium Green-1, Oregon Green, and 488 BAPTA. After activation of the receptors with agonist ligands the emission elicited by the change of intracellular Ca²⁺ concentration can be measured by a luminometer, or a fluorescence imager; a preferred example of this is the fluorescence imager plate reader (FLIPR™, Molecular Devices).

Cells expressing the receptor of interest are plated into clear, flat-bottom, black-walled 96-well plates (Costar) at a density of 80,000-150,000 cells per well and allowed to incubate for 48 hr at 5% CO₂, 37° C. The growth medium is aspirated and 100 μL of loading medium containing Fluo-3 dye is added to each well. The loading medium contains: 20 mM HEPES (Sigma), 0.1% BSA (Sigma), dye/pluronic acid mixture (e.g. 1 mM Fluo-3/AM (Molecular Probes) and 10% pluronic acid (Molecular Probes) mixed immediately before use), and 2.5 mM probenecid (Sigma) (prepared fresh). The cells are allowed to incubate for about 1 hour at 5% CO₂, 37° C.

The compounds of interest are diluted in wash buffer (Hank's BSS (without phenol red), 20 mM HEPES, 2.5 mM probenecid) to a 4× final concentration and aliquoted into a clear v-bottom plate (Nunc). Following the dye incubation, the cells are washed 4 times to remove excess dye using a Denley plate washer. 100 μL final volume of wash buffer is then added to each cell well. Compounds are added to the cell plates and responses are measured using the FLIPR™ instrument. The data are then collected and analyzed using the FLIPR™ software and Graphpad Prism.

Antagonist ligands are identified by the inhibition of the signal elicited by agonist ligands.

GTPγS Functional Assay

Membranes from cells expressing the receptor are suspended in assay buffer (e.g., 50 mM Tris, 100 mM NaCl, 5 mM MgCl₂, 10 μM GDP, pH 7.4) with or without protease inhibitors (e.g., 0.1% bacitracin). Membranes are incubated on ice for 20 minutes, transferred to a 96-well Millipore microtiter GF/C filter plate and mixed with GTPγ³⁵S (e.g., 250,000 cpm/sample, specific activity ˜1000 Ci/mmol) plus or minus unlabeled GTPγS (final concentration=100 μM). Final membrane protein concentration≈90 μg/ml. Samples are incubated in the presence or absence of test compounds for 30 min. at room temperature, then filtered on a Millipore vacuum manifold and washed three times with cold (4° C.) assay buffer. Samples collected in the filter plate are treated with scintillant and counted for ³⁵S in a Trilux (Wallac) liquid scintillation counter. It is expected that optimal results are obtained when the receptor membrane preparation is derived from an appropriately engineered heterologous expression system, i.e., an expression system resulting in high levels of expression of the receptor and/or expressing G-proteins having high turnover rates (for the exchange of GDP for GTP). GTPγS assays are well-known to those skilled in the art, and it is contemplated that variations on the method described above, such as are described by Tian et al. (1994) or Lazareno and Birdsall (1993), may be used.

Microphysiometric Assay

Because cellular metabolism is intricately involved in a broad range of cellular events (including receptor activation of multiple messenger pathways), the use of microphysiometric measurements of cell metabolism can in principle provide a generic assay of cellular activity arising from the activation of any orphan receptor regardless of the specifics of the receptor's signaling pathway.

General guidelines for transient receptor expression, cell preparation and microphysiometric recording are described elsewhere (Salon, J. A. and Owicki, J. A., 1996). Typically cells expressing receptors are harvested and seeded at 3×10⁵ cells per microphysiometer capsule in complete media 24 hours prior to an experiment. The media is replaced with serum free media 16 hours prior to recording to minimize non-specific metabolic stimulation by assorted and ill-defined serum factors. On the day of the experiment the cell capsules are transferred to the microphysiometer and allowed to equilibrate in recording media (low buffer RPMI 1640, no bicarbonate, no serum (Molecular Devices Corporation, Sunnyvale, Calif.) containing 0.1% fatty acid free BSA), during which a baseline measurement of basal metabolic activity is established.

A standard recording protocol specifies a 100 μl/min flow rate, with a 2 min total pump cycle which includes a 30 sec flow interruption during which the acidification rate measurement is taken. Ligand challenges involve a 1 min 20 sec exposure to the sample just prior to the first post challenge rate measurement being taken, followed by two additional pump cycles for a total of 5 min 20 sec sample exposure. Typically, drugs in a primary screen are presented to the cells at 10 μM final concentration.

Follow up experiments to examine dose-dependency of active compounds are then done by sequentially challenging the cells with a drug concentration range that exceeds the amount needed to generate responses ranging from threshold to maximal levels. Ligand samples are then washed out and the acidification rates reported are expressed as a percentage increase of the peak response over the baseline rate observed just prior to challenge.

MAP Kinase Assay

MAP kinase (mitogen activated kinase) may be monitored to evaluate receptor activation. MAP kinase is activated by multiple pathways in the cell. A primary mode of activation involves the ras/raf/MEK/MAP kinase pathway. Growth factor (tyrosine kinase) receptors feed into this pathway via SHC/Grb-2/SOS/ras. Gi coupled receptors are also known to activate ras and subsequently produce an activation of MAP kinase. Receptors that activate phospholipase C (such as Gq/G11-coupled) produce diacylglycerol (DAG) as a consequence of phosphatidyl inositol hydrolysis. DAG activates protein kinase C which in turn phosphorylates MAP kinase.

MAP kinase activation can be detected by several approaches. One approach is based on an evaluation of the phosphorylation state, either unphosphorylated (inactive) or phosphorylated (active). The phosphorylated protein has a slower mobility in SDS-PAGE and can therefore be compared with the unstimulated protein using Western blotting. Alternatively, antibodies specific for the phosphorylated protein are available (New England Biolabs) which can be used to detect an increase in the phosphorylated kinase. In either method, cells are stimulated with the test compound and then extracted with Laemmli buffer. The soluble fraction is applied to an SDS-PAGE gel and proteins are transferred electrophoretically to nitrocellulose or Immobilon. Immunoreactive bands are detected by standard Western blotting technique. Visible or chemiluminescent signals are recorded on film and may be quantified by densitometry.

Another approach is based on evaluation of the MAP kinase activity via a phosphorylation assay. Cells are stimulated with the test compound and a soluble extract is prepared. The extract is incubated at 30° C. for 10 min with gamma-³²P-ATP, an ATP regenerating system, and a specific substrate for MAP kinase such as phosphorylated heat and acid stable protein regulated by insulin, or PHAS-I. The reaction is terminated by the addition of H₃PO₄ and samples are transferred to ice. An aliquot is spotted onto Whatman P81 chromatography paper, which retains the phosphorylated protein. The chromatography paper is washed and counted for ³²P in a liquid scintillation counter. Alternatively, the cell extract is incubated with gamma-³²P-ATP, an ATP regenerating system, and biotinylated myelin basic protein bound by streptavidin to a filter support. The myelin basic protein is a substrate for activated MAP kinase. The phosphorylation reaction is carried out for 10 min at 30° C. The extract can then by aspirated through the filter, which retains the phosphorylated myelin basic protein. The filter is washed and counted for ³²P by liquid scintillation counting.

Cell Proliferation Assay

Receptor activation of the orphan receptor may lead to a mitogenic or proliferative response which can be monitored via ³H-thymidine uptake. When cultured cells are incubated with ³H-thymidine, the thymidine translocates into the nuclei where it is phosphorylated to thymidine triphosphate. The nucleotide triphosphate is then incorporated into the cellular DNA at a rate that is proportional to the rate of cell growth. Typically, cells are grown in culture for 1-3 days. Cells are forced into quiescence by the removal of serum for 24 hrs. A mitogenic agent is then added to the media. 24 hrs later, the cells are incubated with ³H-thymidine at specific activities ranging from 1 to 10 μCi/ml for 2-6 hrs. Harvesting procedures may involve trypsinization and trapping of cells by filtration over GF/C filters with or without a prior incubation in TCA to extract soluble thymidine. The filters are processed with scintillant and counted for ³H by liquid scintillation counting. Alternatively, adherent cells are fixed in MeOH or TCA, washed in water, and solubilized in 0.05% deoxycholate/0.1 N NaOH. The soluble extract is transferred to scintillation vials and counted for ³H by liquid scintillation counting.

Alternatively, cell proliferation can be assayed by measuring the expression of an endogenous or heterologous gene product, expressed by the cell line used to transfect the orphan receptor, which can be detected by methods such as, but not limited to, florescence intensity, enzymatic activity, immunoreactivity, DNA hybridization, polymerase chain reaction, etc.

Promiscuous Second Messenger Assays

It is not possible to predict, a priori and based solely upon the GPCR sequence, which of the cell's many different signaling pathways any given receptor will naturally use. It is possible, however, to coax receptors of different functional classes to signal through a pre-selected pathway through the use of promiscuous G_{α} subunits. For example, by providing a cell based receptor assay system with an endogenously supplied promiscuous G_{α} subunit such as G_{α15} or G_{α≠}or a chimeric G_{α} subunit such as G_{αqz}, a GPCR, which might normally prefer to couple through a specific signaling pathway (e.g., Gₛ, Gᵢ, G_{q}, G₀, etc.), can be made to couple through the pathway defined by the promiscuous G_{α} subunit and upon agonist activation produce the second messenger associated with that subunit's pathway. In the case of G_{α15}, G_{α16} and/or G_{αqz} this would involve activation of the G_{q} pathway and production of the second messenger IP₃. Through the use of similar strategies and tools, it is possible to bias receptor signaling through pathways producing other second messengers such as Ca⁺⁺, cAMP, and K⁺ currents, for example (Milligan and Rees, 1999).

It follows that the promiscuous interaction of the exogenously supplied G_{α} subunit with the receptor alleviates the need to carry out a different assay for each possible signaling pathway and increases the chances of detecting a functional signal upon receptor activation.

Methods for Recording Currents in Xenopus Oocytes

Oocytes were harvested from Xenopus laevis and injected with mRNA transcripts as previously described (Quick and Lester, 1994; Smith et al.,1997). Synthetic, full-length RNA transcripts for human SNORF44 were produced by digestion of the plasmid BN16 with HINDIII followed by transcription with T7 RNA Polymerase (“Message Machine”, Ambion). Oocytes were injected with 15 ng hSN44 receptors synthetic RNA and incubated for 3-4 days at 17 degrees. Dual electrode voltage clamp (Axon Instruments Inc.) was performed using 3 M KCl-filled glass microelectrodes having resistances of 1-2 Mohm. Unless otherwise specified, oocytes were voltage clamped at a holding potential of −80 mV. During recordings, oocytes were bathed in continuously flowing (1-3 ml/min) medium containing 96 mM NaCl, 2 mM KCl, 1.8 mM CaCl₂, 1 mM MgCl₂, and 5 mM HEPES, pH 7.5 (ND96 Buffer). Drugs were applied by local perfusion from a 10 ml glass capillary tube fixed at a distance of 0.5 mm from the oocyte.

Heterologous expression of GPCRs in Xenopus oocytes has been widely used to determine the identity of signaling pathways activated by agonist stimulation (Gundersen et al., 1983; Takahashi et al., 1987). Activation of the phospholipase C (PLC) pathway is assayed by applying test compound in ND96 solution to oocytes previously injected with mRNA for the mammalian receptor and observing inward currents at a holding potential of −80 mV. The appearance of currents that reverse at −25 mV and display other properties of the Ca⁺⁺-activated Cl⁻ (chloride) channel is indicative of mammalian receptor-activation of PLC and release of IP3 and intracellular Ca⁺⁺. Such activity is exhibited by GPCRs that couple to G_{q}.

Inositol Phosphate Assay

Human SNORF44 receptor-mediated activation of the inositol phosphate (IP) second messenger pathways were assessed by radiometric measurement of IP products.

For example, in a 96 well microplate format assay, COS-7 cells expressing the receptor of interest were plated at a density of 70,000 cells per well and allowed to incubate for 24 hours. The cells were then labeled with 0.5 μCi [³H]myo-inositol overnight at 37° C., 5% CO₂. Immediately before the assay, the medium was removed and replaced with 180 μL of Phosphate-Buffered Saline (PBS) containing 10 mM LiCl. The plates were then incubated for 20 min at 37° C., 5% CO₂. Following the incubation, the cells were challenged with agonist (20 μl/well; 10× concentration) for 30 min at 37° C. The challenge was terminated by the addition of 100 μL of 5% v/v trichloroacetic acid, followed by incubation at 4° C. for greater than 30 minutes. Total IPs were isolated from the lysate by ion exchange chromatography. Briefly, the lysed contents of the wells were transferred to a Multiscreen HV filter plate (Millipore) containing Dowex AG1-X8 (200-400 mesh, formate form). The filter plates were prepared adding 100 μL of Dowex AG1-X8 suspension (50% v/v, water:resin) to each well. The filter plates were placed on a vacuum manifold to wash or elute the resin bed. Each well was first washed 2 times with 200 μl of 5 mM myo-inositol. Total [³H]inositol phosphates were eluted with 75 μl of 1.2M ammonium formate/0.1M formic acid solution into 96-well plates. 200 μL of scintillation cocktail was added to each well, and the radioactivity was determined by liquid scintillation counting.

Membrane Preparations

Cell membranes expressing the receptor protein of this invention are useful for certain types of assays including but not restricted to ligand binding assays, GTP-γ-S binding assays, and others. The specifics of preparing such cell membranes may in some cases be determined by the nature of the ensuing assay but typically involve harvesting whole cells and disrupting the cell pellet by sonication in ice cold buffer (e.g. 20 mM Tris HCl, mM EDTA, pH 7.4 at 4° C.). The resulting crude cell lysate is cleared of cell debris by low speed centrifugation at 200×g for 5 min at 4° C. The cleared supernatant is then centrifuged at 40,000×g for 20 min at 4° C., and the resulting membrane pellet is washed by suspending in ice cold buffer and repeating the high speed centrifugation step. The final washed membrane pellet is resuspended in assay buffer. Protein concentrations are determined by the method of Bradford (1976) using bovine serum albumin as a standard. The membranes may be used immediately or frozen for later use.

Generation of Baculovirus

The coding region of DNA encoding the human receptor disclosed herein may be subcloned into pBlueBacIII into existing restriction sites or sites engineered into sequences 5′ and 3′ to the coding region of the polypeptides. To generate baculovirus, 0.5 μg of viral DNA (BaculoGold) and 3 μg of DNA construct encoding a polypeptide may be co-transfected into 2×10⁶ Spodoptera frugiperda insect Sf9 cells by the calcium phosphate co-precipitation method, as outlined by Pharmingen (in “Baculovirus Expression Vector System: Procedures and Methods Manual”). The cells then are incubated for 5 days at 27° C.

The supernatant of the co-transfection plate may be collected by centrifugation and the recombinant virus plaque purified. The procedure to infect cells with virus, to prepare stocks of virus and to titer the virus stocks are as described in Pharmingen's manual.

Radiolabeled Ligand Binding Assays

Cells expressing the receptors of this invention may be used to screen for ligands for said receptors, for example, by [¹²⁵I]radioligand binding assays. The same assays may be used to identify agonists or antagonists of the receptor that may be employed for a variety of therapeutic purposes.

Radioligand binding assays are performed by diluting membranes prepared from cells expressing the receptor in 50 mM Tris buffer (pH=7.4 at 0° C.) containing 0.1% bovine serum albumin (Sigma), aprotinin (0.005 mg/ml, Boehringer Mannheim) and bestatin (0.1 mM, Sigma) as protease inhibitors. The final protein concentration in the assay is 12-40 μg/ml. Membranes are then incubated with [¹²⁵I]-labeled surrogate ligand (NEN, specific activity 2200 Ci/mmole) in the presence or absence of competing ligands on ice for 60 min in a total volume of 250 μl in 96 well microtiter plates. The bound ligand is separated from free by filtration through GF/B filters presoaked in 0.5% polyethyleneimine (PEI), using a Tomtec (Wallac) vacuum filtration device. After addition of Ready Safe (Beckman) scintillation fluid, bound radioactivity is quantitated using a Trilux (Wallac) scintillation counter (approximately 40% counting efficiency of bound counts). Data is fit to non-linear curves using GraphPad Prism.

In this manner, agonist or antagonist compounds that bind to the receptor may be identified as they inhibit the binding of the labeled ligand to the membrane protein of cells expressing the said receptor. Non-specific binding was defined as the amount of radioactivity remaining after incubation of membrane protein in the presence of 100 nM of the unlabeled ligand corresponding to the radioligand used. In equilibrium saturation binding assays membrane preparations or intact cells transfected with the receptor are incubated in the presence of increasing concentrations of the labeled compound to determine the binding affinity of the labeled ligand. The binding affinities of unlabeled compounds may be determined in equilibrium competition binding assays, using a fixed concentration of labeled compound in the presence of varying concentrations of the displacing ligands.

Localization of mRNA Coding for Human SNORF44.

Ouantitative PCR using a fluorogenic probe with real time detection: Quantitative PCR using fluorogenic probes was used to characterize the distribution of SNORF44 RNA. This assay utilizes two oligonucleotides for conventional PCR amplification and a third specific oligonucleotide probe that is labeled with a reporter at the 5′ end and a quencher at the 3′ end of the oligonucleotide. In the current invention, FAM (6-carboxyfluorescein) was used as the reporter, and a Black Hole™ quencher (Biosearch Corp, Novato Calif.). As amplification progresses, the labeled oligonucleotide probe hybridizes to the gene sequence between the two oligonucleotides used for amplification. The nuclease activity of Taq thermostable DNA polymerase is utilized to cleave the labeled probe. This separates the quencher from the reporter and generates a fluorescent signal that is directly proportional to the amount of amplicon generated. This labeled probe confers a high degree of specificity. Non-specific amplification is not detected as the labeled probe does not hybridize and as a consequence is not cleaved. All experiments were conducted in a PE7700 Sequence Detection System (Applied Biosystems, Foster City Calif.),

Ouantitative RT-PCR: Quantitative RT-PCR was used for the detection of SNORF44 RNA. For use as a template in quantitative PCR reactions, cDNA was synthesized by reverse transcription from total human RNA. Reverse transcription by SuperScriptII RNAse H⁻ and (GibcoBRL/life Technologies) was primed using random hexamers. To assay for potential genomic DNA contamination, parallel reactions were carried out in the absence of reverse transcriptase. Another set of parallel reactions included 32P labeled dCTP to allow quantification of the cDNA. Following reverse transcription, cDNA was phenol/chloroform extracted and precipitated. Incorporation of 32P dCTP was assessed after precipitation with trichloroacetic acid and the amount of cDNA synthesized was calculated.

For PCR reactions primers with the following oligonucleotide sequences were used:

Human SNORF44:

Forward primer

| | |
|---|---|
| SNORF44H.seq-367F | (SEQ ID NO: 27) |

5′-CGTGCTCATCTTCGCCCT-3′
Reverse primer

| | |
|---|---|
| SNORF44H.seq-443R | (SEQ ID NO: 28) |

5′-TGCGCATGGCCTTGCT-3′
Fluorogenic oligonucleotide probe:

| | |
|---|---|
| SNORF44H.seg-387T | (SEQ ID NO: 29) |

5′ (6-FAM)-CGCTCTTTGGCAATGCTCTGGTGTTCTA-(BH) 3′

Using this primer set, amplicon length is 76 bp for SNORF44. Each PCR reaction contained 3.0 ng cDNA. Oligonucleotide concentrations were: 500 nM of forward and reverse primers, and 50 nM of fluorogenic probe. PCR reactions were carried out in 50 μl volumes using TaqMan universal PCR master mix (Applied Biosystems, Foster City, Calif.)). Buffer for RT-PCR reactions contained a fluor used as a passive reference (ROX: Applied Biosystems proprietary passive reference I). All reagents for PCR (except cDNA and oligonucleotide primers) were obtained from Applied Biosystems (Foster City, Calif.). Reactions were carried in a PE7700 sequence detection system (Applied Biosystems) using the following thermal cycler profile: 50° C. 2 min., 95° C. 10 min., followed by 40 cycles of: 95° C., 15 sec., 60° C. 1 min. Standard curves for quantification of human SNORF 44 were constructed using genomic DNA. Negative controls consisted of cDNA blanks. To confirm that the RNA was not contaminated with genomic DNA, PCR reactions were carried out without reverse transcription using Taq DNA polymerase.

Integrity of RNA was assessed by amplification of RNA coding for cyclophilin or glyceraldehyde 3-phosphate dehydrogenase (GAPDH). Following reverse transcription and PCR amplification, data was analyzed using Applied Biosystems sequence detection software. The fluorescent signal from each well was normalized using an internal passive reference, and data was fitted to a standard curve to obtain copy number of SNORF44 expressed.

### RESULTS AND DISCUSSION

Isolation of a Full-Length Human SNORF44 Receptor

MOPAC (Mixed Oligonucleotide Primed Amplification of cDNA) was carried out using degenerate primers based on alignments of the sixth and seventh transmembrane domains of more than 180 members of the rhodopsin superfamily of G protein coupled receptors to amplify human hypothalamic cDNA. Sequence analysis of the PCR products revealed that one PCR product(MPR133-HUMHT-091) was likely to be a novel G protein-coupled receptor-like sequence fragment (comprising the region between the sixth and seventh transmembrane domains) based on its homology to other known G protein-coupled receptors. This novel sequence was designated hSNORF44 (human SNORF44). Through BLAST and GRAIL analysis, the DNA sequence corresponding to the region of the SNORF44 gene coding for the third transmembrane domain through a stop codon following the seventh transmembrane domain was found to be represented in a large human genomic DNA entry in (GenBank accession number AC005961) which was not annotated as containing a GPCR-encoding gene. The partial SNORF44 sequence represented in AC005961 was interrupted by four introns, so that the coding sequence for the region of the gene encoding the third through the seventh transmembrane domains spanned more than 10,000 bp. The sequence inferred from genomic DNA corresponding to the third transmembrane domain through a putative stop codon after the seventh transmembrane domain was confirmed by sequence analysis of PCR products amplified from human spinal cord. Rapid Amplification of cDNA ends (RACE) was used to obtain the sequence of the 5′ end of the receptor. This method generated sequence information for SNORF44 from the 5′ untranslated region upstream from a potential initiating methionine to the coding sequence of the third transmembrane domain. The full-length coding sequence of SNORF44 was determined by combining the sequence information from 5′ and 3′ fragments described above. The entire coding region was then amplified by PCR from human spinal cord cDNA and subcloned into pcDNA3.1(−) for expression and functional analyses. A human SNORF44 clone representing the consensus sequence of several PCR products was constructed in pcDNA3.1(−) and this construct was designated BN16.

The putative amino acid sequence encoded by the full-length human SNORF44 receptor sequence is most closely related to NPFF2 (30% identity), and the NPY receptors Y1, Y2, and Y4 (28-29% identity), suggesting that SNORF44 may represent a novel peptide receptor. Hydrophobicity (Kyte-Doolittle) analysis of the amino acid sequence of the full length clone indicates the presence of seven hydrophobic regions, consistent with the seven transmembrane domains of a G protein coupled receptor (FIGS. 2A-2B). There are two potential N-linked glycosylation sites: one in the N-terminus at Asn19 and one in the first extracellular loop at Asn106. Human SNORF44 also contains three potential casein kinase II phosphorylation sites in the C-terminal tail (S377, T386, and S425) as well as two potential protein kinase C phosphorylation sites at T158 and S377 in the second intracellular loop and in the C-terminal tail, respectively.

Increase in Intracellular Ca²⁺ Release

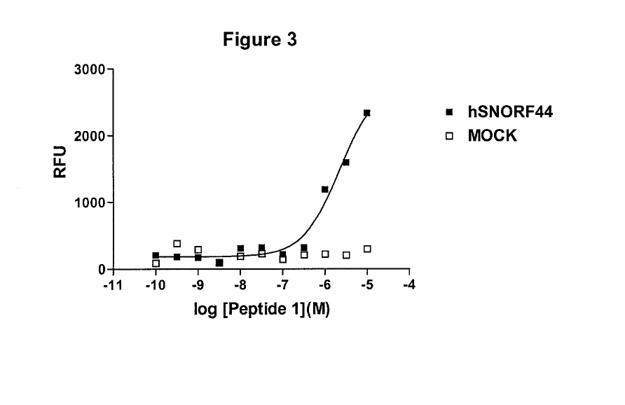

COS-7 cells were transiently transfected with hSNORF44 or vector DNA (mock) as described in Materials and Methods and screened against a variety of potential ligands. Only application of Peptide 1 resulted in concentration-dependent release of intracellular Ca²⁺ (as measured by FLIPR™) in COS-7 cells transfected with SNORF44 (FIG. 3). Additional screening with compounds structurally related to Peptide 1 revealed two additional compounds, Peptide 2 and Peptide 3, that stimulated release of intracellular Ca²⁺ in cells transfected with SNORF44 as measured by FLIPR™ (data not shown). In contrast, neither NPFF nor NPY (ligands activating the most closely related GPCRs) had any significant effect on intracellular Ca²⁺ release in vector-transfected cells (data not shown). The three active compounds are peptides whose sequences share a conserved Arginine with NPFF and NPY peptides at the penultimate C-terminal position. Their sequences are:

| | | |
|---|---|---|
| Peptide 1: | YSMEHFRW-NH2 | (SEQ ID NO: 30) |
| Peptide 2: | MGHFRF-NH2 | (SEQ ID NO: 31) |
| Peptide 3: | YGGHFRW-NH2 | (SEQ ID NO: 32) |

The activities of these three compounds were characterized in additional functional assays (see below) to more precisely determine their potencies and signalling properties at SNORF44.

Peptide Synthesis:

Abbreviations: Fmoc: 9-Fluorenyloxycarbonyl-; Trityl: triphenylmethyl-; tBu-: tertiary butyl ester; OtBu-: tertiary butyl ether; N^{g}: N-guanidinyl; Nⁱⁿ: N-Indole; MBHA: methylbenzhydlamine; DMF: N,N-dimethylformamide; NMP: N-Methylpyrrolidinone; DIEA: diisopripylethyl amine; TFA: trifluoroacetic acid.

Small scale peptide syntheses were performed either manually, by using a sintered glass column with argon pressure to remove solvents and reagents, or by using an Advanced ChemTech 396-9000 automated peptide synthesizer (Advanced ChemTech, Louisville, Ky.). Large scale peptide syntheses were performed on a CS Bio 536 (CS Bio Inc., San Carlos, Calif.). Fmoc-Alanine-OH, Fmoc-Cysteine(Trityl)-OH, Fmoc-Aspartic acid(tBu)-OH, Fmoc-Glutamic acid(tBu)-OH, Fmoc-Phenylalanine-OH, Fmoc-Glycine-OH, Fmoc-Histidine(Trityl)-OH, Fmoc-Isoleucine-OH, Fmoc-Lysine(Boc)-OH, Fmoc-Leucine-OH, Fmoc-Methionine-OH, Fmoc-Asparagine(Trityl)-OH, Fmoc-Proline-OH, Fmoc-Glutamine(Trityl)-OH, Fmoc-Arginine(N^{g}-2,2,4,6,7-Pentamethyldihydrobenzofuran-5-sulfonyl)-OH, Fmoc-Serine(OtBu-OH, Fmoc-Threonine(OtBu)-OH, Fmoc-Valine-OH, Fmoc-Tryptophan(NⁱⁿBoc)-OH, Fmoc-Tyrosine(OtBu)-OH, Fmoc-Cyclohexylalanine-OH, and Fmoc-Norleucine, Fmoc-O-benzyl-phosphotyrosine were used as protected amino acids. Any corresponding D-amino acids had the same side-chain protecting groups, with the exception of Fmoc-D-Arginine, which had a N^{g}-2,2,5,7,8-pentamethylchroman-6-sulfonyl protecting group.

Peptides with C-terminal amides were synthesized on solid phase using Rink amide-MBHA resin. The Fmoc group of the Rink Amide MBHA resin was removed by treatment with 30% piperidine in DMF for 5 and 30 minutes respectively. After washing with DMF (3 times), methanol (2 times) and DMF/NMP (3 times), the appropriate Fmoc-protected amino acid (4 eq.) was coupled for 2 hours with HBTU or HATU (4 eq.) as the activating agent and DIEA (8 eq.) as the base. In manual syntheses, the ninhydrin test was used to test for complete coupling of the amino acids. The Fmoc groups were removed by treatment with 30% piperidine in DMF for 5 and 30 minutes respectively. After washing with DMF (3 times), methanol (2 times) and DMF/NMP (3 times), the next Fmoc-protected amino acid (4 eq.) was coupled for 2 hours with HBTU or HATU (4 eq.) as the activating agent and DIEA (8 eq.) as the base. This process of coupling and deprotection of the Fmoc group was continued until the desired peptide was assembled on the resin. The N-terminal Fmoc group was removed by treatment with 30% piperidine in DMF for 5 and 30 minutes respectively. After washing with DMF (3 times), methanol (2 times), the resin(s) was vacuum dried for 2 hours. Cleavage of the peptide-on-resin and removal of the side chain protecting groups was achieved by treating with TFA:ethanedithiol:thioanisole:m-cresol:water:triisopropylsilane:phenol, 78:5:3:3:3:5:3 (5 mL per 100 mg resin) for 2.5-3 hours. The cleavage cocktail containing the peptide was filtered into a round bottom flask and the volatile liquids were removed by rotary evaporation at 30-40° C. The peptides were precipitated with anhydrous ether, collected on a medium-pore sintered glass funnel by vacuum filtration, washed with ether and vacuum dried.

Peptides with C-terminal acids were synthesized using 2-chlorotrityl chloride resin. The first amino acid was attached to the resin by dissolving 0.6-1.2 eq. of the appropriate Fmoc-protected amino acid described above in dichloromethane (a minimal amount of DMF was added to facilitate the dissolution if necessary). To this was added DIEA (4 eq. Relative to the Fmoc-amino acid) and the solution was added to the resin and shaken for 30-120 minutes. The solvents and the excess reagents were drained and the resin was washed with dichloromethane:methanol:DIEA (17:2:1) (3 times), dichloromethane (3 times), DMF (2 times), dichloromethane (2 times), and vacuum dried. The process of deprotection of the Fmoc group and coupling the appropriate Fmoc-protected amino acid was continued as described above, until the desired, fully protected peptide was assembled on the resin. The process for removal of the final Fmoc group and the cleavage and deprotection of the peptides was the same as described above for the peptides with C-terminal amides.

Purification of the peptides was achieved by preparative High performance Column Chromatography (HPLC), using a reverse-phase C-18 column (25×250 mm) (Primesphere or Vydac) with a gradient of acetonitrile (0.1% TFA) in water (0.1% TFA). The general gradient was from 10%-90% acetonitrile in water over 40 minutes. The fractions corresponding to each peak on the HPLC trace was collected, freeze dried and analyzed by electrospray mass spectrometery. The fraction having the correct mass spectral data corresponding to the desired peptide was then further analyzed by amino acid analysis, if necessary. All purified peptides were tested for homogeneity by analytical HPLC using conditions similar to that described above, but by using a 2.5×250 mm analytical column, and generally were found to have >95% purity.

Inositol Phosphate (IP) Release

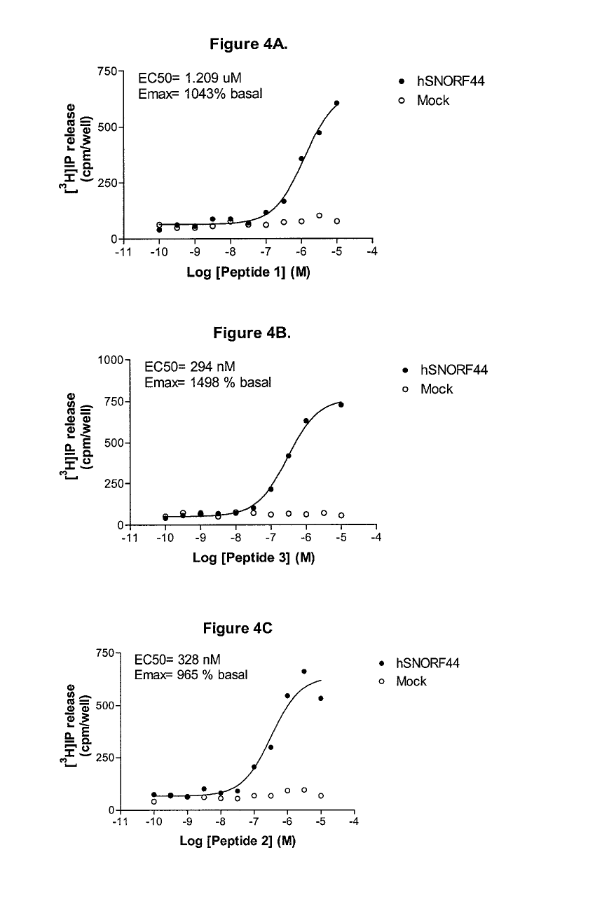

Exposure of hSNORF44-transfected COS-7 cells (but not mock-transfected cells) to Peptide 1 caused the dose-dependent release of IP second messengers (approximately 10-fold above basal) with an EC50 of 1.21 uM (FIG. 4A). Peptide 2 (FIG. 4B) and Peptide 3 (FIG. 4C) were more potent agonists in this assay with EC50's=294 nM and 328 nM, respectively. The potencies of these peptides in stimulating SNORF44 are somewhat lower than would be expected for an endogenous peptide ligand. While the possibility exists that one or more of these peptides activates SNORF44 in vivo, their reduced potencies compared with those of endogenous peptide ligands for their cognate receptors suggest that they are not endogenous ligands for SNORF44. However, they are agonists since they robustly and reproducibly activate SNORF44, mimicking the activity of any endogenous ligand, and must contain structural components that trigger receptor activation. Such agonists can be substituted for an endogenous ligand in a wide range of assays including functional assays and binding assays, and can be used in screening for chemical compounds (agonists and antagonists) that interact with SNORF44. Such agonists can also be used to define receptor-effector coupling mechanisms used by the receptor. In this case, the ability of these three agonists to stimulate intracellular calcium release and inositol phospholipid hydrolysis in cells transfected with SNORF44 indicates that SNORF44 couples to phospholipase C stimulation via a Gq-type G-protein in COS-7 cells.

Activation of Calcium-Activated Cl⁻ Currents in Human SNORF44-Expressing Xenopus Oocytes

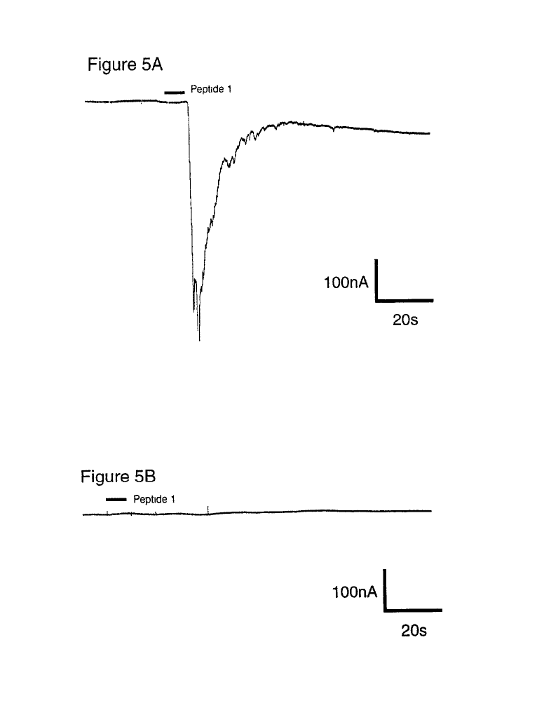

In Xenopus laevis oocytes injected with SNORF44 mRNA, Peptide 1 elicited oscillatory Cl− currents through G protein-coupled stimulation of the phosphoinositide/Ca2+ second messenger system, which in turn leads to the activation of a Ca2+-dependent Cl− current. As shown in FIG. 5B, control oocytes, lacking injection of foreign mRNA, typically showed no response to Peptide 1 (n=5). However, in oocytes injected with SNORF44 mRNA, the current amplitude averaged 1036±306 nA (n=10) in response to 10 μM Peptide 1 (FIG. 5A). Peptide 2 and Peptide 3 (10 μM) also elicited a strong response [241±65 nA (n=4) and 250±46 nA (n=8), respectively] from oocytes injected with SNORF44 mRNA, but not in control oocytes (data not shown).

Detection of RNA Coding for Human SNORF44

RNA was isolated from multiple tissues (listed in Table 1) and assayed as described. Quantitative RT-PCR using a fluorgenic probe demonstrated RNA encoding human SNORF 44 to be localized in highest abundance in the central nervous system. All CNS tissues assayed demonstrate moderate levels of SNORF44 RNA. The broad distribution of SNORF44 RNA implies a modulatory role in multiple systems within the CNS. CNS regions expressing the highest levels of SNORF44 RNA are the: pontine reticular formation, hypothalamus and dorsal root ganglia (Table 1).

Highest levels of SNORF44 RNA are found in the pontine reticular formation suggesting a role in regulation of REM sleep, arousal and superspinal modulation of pain. Equally high levels of SNORF 44 RNA in the dorsal root ganglia strongly imply an important role in sensory transmission or modulation (including nociception). The hypothesis that SNORF44 is involved in regulation of sensory transmission is supported by the presence of moderate levels of SNORF44 RNA in the medulla oblongata. As most afferent sensory input is conducted through the dorsal roots, SNORF44 is well positioned to play a major role is a potential site for drug intervention.

Another region expressing high levels of SNORF44 RNA is the hypothalamus. This suggests a role for SNORF44 in neuroendocrine regulation, regulation of circadian rhythms, regulation of appetite/feeding behavior, regulation of sexual behavior, and other functions that are modulated by the hypothalamus.

The hippocampal formation and amygdala contain moderate levels of SNORF44 RNA. Localization to these structures supports the hypothesis that SNORF44 is involved in the modulation of learning and memory as well as having a role in the regulation of fear and mood, and may provide a target for the treatment of depression, anxiety, phobias and mood disorders.

In peripheral tissues, the highest level of SNORF44 RNA is found in the kidney (both cortex and medulla). However, levels in peripheral tissue in general are much lower than those found in the CNS. Expression in the kidney suggests a role in fluid and electrolyte balance, and has a potential role in the treatment of hypertension. Other tissues assayed contain low levels of SNORF44 RNA as indicated in Table 1.

In summary, the distribution of human SNORF44 RNA implies broad regulatory function in the CNS, most notably in sensory transmission, modulation of the limbic system, and modulation of feeding/circadian rhythms. Its presence, albeit at low levels, in peripheral tissues implies a broad regulatory role in multiple organ systems.

In addition to the potential therapeutic applications identified in Table 1, the localization data for mRNA encoding the human SNORF44 receptor indicates that the DNA encoding the human SNORF44 receptor can be used to predict the likelihood that a tissue sample of unknown tissue origin is of pontine reticular formation origin with respect to a given individual. In addition, with respect to a given individual, one could determine whether a given tissue sample of unknown origin is of pontine reticular formation origin as opposed to having the origin of another tissue, e.g. the liver, spleen, or lung. Such determinations may be used for various purposes including but not limited to the detection of tumor metastasis.

TABLE 1
Summary of distribution of mRNA coding for human SNORF44.

| | Copy | |
|---|---|---|
| | number/ng | |
| TISSUE | cDNA | Potential applications |
| adipose | not | Metabolic disorders |
| tissue | detected | |
| adrenal gland | 44 | Regulation of metabolic |
| (whole) | | steroids, regulation of |
| | | epinephrine release |
| amygdala | 417 | Depression, phobias, |
| | | anxiety, mood disorders |
| cerebellum | 217 | Motor coordination |
| cerebral | 359 | Cognition, sensory and motor |
| cortex | | integration. |
| dorsal root | 1438 | Sensory transmission, pain |
| ganglia | | |
| heart | 621 | Cardiovascular indications |
| hippocampus | 352 | Cognition/memory |
| hypothalamus | 1577 | Appetite/obesity, |
| | | neuroendocrine regulation |
| kidney cortex | 595 | Hypertension, electrolyte |
| | | balance |
| kidney | 623 | Hypertension, electrolyte |
| medulla | | balance |
| liver | not | Diabetes |
| | detected | |
| lung | Trace | Respiratory disorders, |
| | | asthma |
| medulla | 318 | Sensory transmission/ |
| | | integration, cardiovascular |
| | | regulation, respiratory |
| | | regulation, |
| pancreas | Trace | Diabetes, endocrine |
| | | disorders |
| pituitary | 209 | Endocrine/ neuroendocrine |
| (whole) | | regulation |
| pontine | 2178 | Sleep disorders, epilepsy |
| reticular | | |
| formation | | |
| prostate | 99 | Benign prostatic |
| gland | | hypertrophy, sexual |
| | | disfunction |
| skeletal | not | Musculoskeletal disorders |
| muscle | detected | |
| small | Trace | Gastrointestinal disorders |
| intestine | | |
| spinal cord | 213 | Analgesia, sensory |
| lumbar | | modulation and transmission |
| spleen | Trace | Immune disorders |
| stomach | 123 | Gastrointestinal disorders |
| testes | 88 | reproductive disorders, |
| | | regulation of steroid |
| | | hormones |
| thalamus | 294 | Sensory integration |
| | | disorders |
| urinary | 67 | Urinary incontinance |
| bladder | | |
| (whole) | | |
| uterus | 35 | Reproductive disorders, |
| | | dysmenorrhea |

Discussion

The amino acid sequence of the three peptides that can activate intracellular calcium and phosphoinositide metabolism in COS-7 cells expressing SNORF44 accumulation Peptide 1, Peptide 2 and Peptide 3 share significant similarity between them. Their main features are the core sequence His-Phe-Arg, ending at the carboxyl terminus by an aromatic amino acid either phenylalanine or tryptophan. This core motif is contained in endogenous peptides derived from the common precursor pro-opio-melanocortin (POMC). Moreover, the exact sequence of Peptide 1 is specifically contained in the sequence of gamma melanocortin (γ-MSH). Earlier studies have demonstrated that tissues that express POMC, such as human pituitary, brain, and some tumors actually process this precursor originating two distinct γ-MSH fragments (Nakanishi, S. et al, 1980; Benjannet, S. et al, 1980; Shibasaki, T. et al, 1980). The finding that γ-MSH immunoreactivity is present in secretory granules of rat pituitary corticotrophs together with adrenocorticotropin (ACTH) and β-lipotropin (β-LPH), and that γ-MSH immunoreactivity is present in human plasma, indicates that this peptide is released to the circulation consistent with a role as a messenger with hormonal activity. Besides this potential hormonal role γ-MSH has also been identified in various regions of the rat brain, where it is present in two main systems one with cell bodies in the arcuate nucleus of the hypothalamus with projections to the forebrain, diencephalon and brain stem; and another in the nucleus commisuralis (Kawai, Y. et al, 1984). The CNS localization of the γ-MSH correlates well with its behavioral effects. The administration of γ-MSH has been shown to elicit behavioral activity opposite to that of ACTH, to attenuate the antinociception, hypothermia, and the release of α-MSH elicited by β-endorphin, and to decrease the acquisition of heroin self-administration (van Ree, J. M. et al, 1981). γ-MSH is an agonist of two of the five recombinant melanocortin (MC) receptors, MC3 and MC4 (Schioth, H. B. et al, 1996). However, by means of the use of selective MC3/MC4 receptor antagonist it has been possible to identify in vivo pharmacological effects of γ-MSH that are not mediated by the activation of MC receptors, such as, increase in blood pressure, tachycardia, antinociception, and catalepsy (Varga, K. et al, 1996; Nijsen, M. J. et al, 2000; Klusa, V. at al, 2001). The structure activity relationship of the cardiovascular effects mediated by analogs of γ-MSH indicates that the shortest active fragment is His-Phe-Arg-Trp the sequence contained in the peptide fragments that were found to activate SNORF44 (Van Bergen, P. et al, 1995; Van Bergen, P. et al, 1997). This pharmacological evidence altogether suggests that peptide sequences contained in γ-MSH are endogenous ligands of the SNORF44 receptor, and in turn that the in vivo effects of these peptides are mediated by the SNORF44 receptor.

### REFERENCES

Bradford, M. M., “A rapid and sensitive method for the quantitation of microgram quantities of protein utilizing the principle of protein-dye binding”, Anal. Biochem. 72: 248-254 (1976).

Benjannet, S., et al. Nature 285: 415-6 (1980).

Burns, C. M., et al. Nature 387(6630: 303-308 (1997).

Bush, et al., “Nerve growth factor potentiates bradykinin-induced calcium influx and release in PC12 cells” J. Neurochem. 57: 562-574 (1991).

Chu, Y. Y., et al., “Characterization of the rat A2a adenosine receptor gene”, DNA Cell Biol. 15(4): 329-337 (1996).

Dascal, N., et al., “Atrial G protein-activated K⁺ channel: expression cloning and molecular properties” Proc. Natl. Acad. Sci. USA 90: 10235-10239 (1993).

Fong, T. M., et al., “Mutational analysis of neurokinin receptor function” Can. J. Physio. Pharmacol. 73(7): 860-865 (1995).

Graziano, M. P. et al., “The amino terminal domain of the glucagon-like peptide-1 receptor is a critical determinant of subtype specificity” Receptors Channels 4(1): 9-17 (1996).

Guan, X. M., et al., “Determination of Structural Domains for G Protein Coupling and Ligand Binding in β3—Adrenergic Receptor” Mol. Pharmacol. 48(3): 492-498 (1995).

Gundersen, C. B., et al., “Serotonin receptors induced by exogenous messenger RNA in Xenopus oocytes” Proc. R. Soc. Lond. B. Biol. Sci. 219(1214): 103-109 (1983).

Graziano, M. P. et al., “The amino terminal domain of the glucagon-like peptide-1 receptor is a critical determinant of subtype specificity” Receptors Channels 4(1): 9-17 (1996).

Inanobe, A., et al., “Characterization of G-protein-gated K⁺ channels composed of Kir3.2 subunits in dopaminergic neurons of the substantia nigra” J. of Neuroscience 19(3): 1006-1017 (1999).

Kawai, Y. et al., Brain Res 297: 21-32 (1984).

Klusa, V., et al., Neuropeptides February;35(1): 50-7 (2001).

Krapivinsky, G., et al., “The cardiac inward rectifier K⁺ channel subunit, CIR, does not comprise the ATP-sensitive K⁺ channel, IKATP” J. Biol. Chem. 270: 28777-28779 (1995b).

Krapivinsky, G., et al., “The G-protein-gated atrial K⁺ channel IKACh is a heteromultimer of two inwardly rectifying K(⁺)-channel proteins” Nature 374: 135-141 (1995).

Kubo, Y., et al., “Primary structure and functional expression of a rat G-protein-coupled muscarinic potassium channel” Nature 364: 802-806 (1993).

Lazareno, S. and Birdsall, N. J. M. “Pharmacological characterization of acetylcholine stimulated [35S]-GTPgS binding mediated by human muscarinic m1-m4 receptors: antagonist studies”, Br. J. Pharmacology 109: 1120-1127 (1993).

Milligan, G. and S. Rees, “Chimaeric G alpha proteins: their potential use in drug discovery” Trends Pharmacol. Sci. 20: 118-124 (1999).

Nakanishi, S., et al., Nature 287: 752-5 (1980).

Nijsen, M. J., et al., Br J Pharmacol 131: 1468-74 (2000).

Quick, M. W. and Lester, H. A., “Methods for expression of excitability proteins in Xenopus oocytes”, Meth. Neurosci. 19: 261-279 (1994).

Sakura, N., et al., “Structure-activity relationships of rat neuromedin U for smooth muscle contraction” Chem Pharm Bull (Tokyo) 39: 2016-2020 (1991).

Salon, J. A. and Owicki, J. A., “Real-time measurements of receptor activity: Application of microphysiometic techniques to receptor biology” Methods in Neuroscience 25: 201-224, Academic Press (1996).

Schioth, H. B., et al., Pharmacol Toxicol 79: 161-165 (1996).

Shibasaki, T., et al., Nature 285: 416-417 (1980).

Smith, K. E., et al., “Expression cloning of a rat hypothalamic galanin receptor coupled to phosphoinositide turnover.” J. Biol. Chem. 272: 24612-24616 (1997).

Spurney, R. F., et al., “The C-terminus of the thromboxane receptor contributes to coupling and desensitization in a mouse mesangial cell line”, J. Pharmacol. Exp. Ther. 283(1): 207-215 (1997).

Takahashi, T., et al., “Rat brain serotonin receptors in Xenopus oocytes are coupled by intracellular calcium to endogenous channels.” Proc. Natl. Acad. Sci. USA 84(14): 5063-5067 (1987).

Tian, W., et al., “Determinants of alpha-Adrenergic Receptor Activation of G protein: Evidence for a Precoupled Receptor/G protein State.” Molecular Pharmacology 45: 524-553 (1994).

Underwood, D. J. et al., “Structural model of antagonist and agonist binding to the angiotensin II, AT1 subtype, G protein coupled receptor”, Chem. Biol. 1(4): 211-221 (1994).

Van Bergen, P., et al., Eur J Pharmacol 294: 795-803 (1995).

Van Bergen, P., et al., Br J Pharmacol 120: 1561-1567 (1997).

van Ree, J. M., et al., Ciba Found Symp 81: 263-276 (1981).

Varga, K., et al., J Neurosci 16: 5182-5188 (1996).

What is claimed is:

1. An isolated nucleic acid encoding a mammalian SNORF44 receptor.

2. The nucleic acid of claim 1, wherein the nucleic acid is DNA.

3. The DNA of claim 2, wherein the DNA is cDNA.

4. The DNA of claim 2, wherein the DNA is genomic DNA.

5. The nucleic acid of claim 1, wherein the nucleic acid is RNA.

6. The nucleic acid of claim 1, wherein the mammalian SNORF44 receptor is a human SNORF44 receptor.

7. The nucleic acid of claim 6, wherein the human SNORF44 receptor has an amino acid sequence identical to that encoded by the plasmid pcDNA3.1-hSNORF44-f (Patent Deposit Designation PTA-100).

8. The nucleic acid of claim 6, wherein the human SNORF44 receptor has an amino acid sequence identical to the amino acid sequence shown in FIGS. 2A-2B (SEQ ID NO: 2).

9. A purified mammalian SNORF44 receptor protein.

10. The purified mammalian SNORF44 receptor protein of claim 9, wherein the mammalian SNORF44 receptor protein is a human SNORF44 receptor protein.

11. A vector comprising the nucleic acid of claim 1 or claim 6.

12. A vector of claim 11 adapted for expression in a cell which comprises the regulatory elements necessary for expression of the nucleic acid in the cell operatively linked to the nucleic acid encoding the receptor so as to permit expression thereof, wherein the cell is a bacterial, amphibian, yeast, insect or mammalian cell.

13. The vector of claim 12, wherein the vector is a baculovirus.

14. The vector of claim 11, wherein the vector is a plasmid.

15. The plasmid of claim 14 designated pcDNA3.1-hSNORF44-f (Patent Deposit Designation PTA-100).

16. A cell comprising the vector of claim 12.

17. A cell of claim 16, wherein the cell is a non-mammalian cell.

18. A cell of claim 17, wherein the non-mammalian cell is a Xenopus oocyte cell or a Xenopus melanophore cell.

19. A cell of claim 16, wherein the cell is a mammalian cell.

20. A mammalian cell of claim 19, wherein the cell is a COS-7 cell, a 293 human embryonic kidney cell, a NIH-3T3 cell, a LM(tk−) cell, a mouse Y1 cell, or a CHO cell.

21. A cell of claim 16, wherein the cell is an insect cell.

22. An insect cell of claim 21, wherein the insect cell is an Sf9 cell, an Sf21 cell or a Trichoplusia ni 5B-4 cell.

23. A membrane preparation isolated from the cell of any one of claims 16, 17, 19, 20, 21 or 22.

24. A nucleic acid probe comprising at least 15 nucleotides, which probe specifically hybridizes with a nucleic acid encoding a mammalian SNORF44 receptor, wherein the probe has a sequence complementary to a unique sequence present within one of the two strands of the nucleic acid encoding the human SNORF44 receptor contained in plasmid pcDNA3.1-hSNORF44-f (Patent Deposit Designation PTA-100).

25. A nucleic acid probe comprising at least 15 nucleotides, which probe specifically hybridizes with a nucleic acid encoding a mammalian SNORF44 receptor, wherein the probe has a sequence complementary to a unique sequence present within (a) the nucleic acid sequence shown in FIGS. 1A-1B (SEQ ID NO: 1) or (b) the reverse complement thereof.

26. The nucleic acid probe of claim 25, wherein the nucleic acid is DNA.

27. The nucleic acid probe of claim 25, wherein the nucleic acid is RNA.

28. An antisense oligonucleotide having a sequence capable of specifically hybridizing to the RNA of claim 5, so as to prevent translation of the RNA.

29. An antisense oligonucleotide having a sequence capable of specifically hybridizing to the genomic DNA of claim 4, so as to prevent transcription of the genomic DNA.

30. An antisense oligonucleotide of claim 28 or 29, wherein the oligonucleotide comprises chemically modified nucleotides or nucleotide analogues.

31. An antibody capable of binding to a mammalian SNORF44 receptor encoded by the nucleic acid of claim 1.

32. An antibody of claim 31, wherein the mammalian SNORF44 receptor is a human SNORF44 receptor.

33. An agent capable of competitively inhibiting the binding of the antibody of claim 31 to a mammalian SNORF44 receptor.

34. An antibody of claim 33, wherein the antibody is a monoclonal antibody or antisera.

35. A pharmaceutical composition comprising (a) an amount of the oligonucleotide of claim 28 capable of passing through a cell membrane and effective to reduce expression of a mammalian SNORF44 receptor and (b) a pharmaceutically acceptable carrier capable of passing through the cell membrane.

36. A pharmaceutical composition of claim 35, wherein the oligonucleotide is coupled to a substance which inactivates mRNA.

37. A pharmaceutical composition of claim 36, wherein the substance which inactivates mRNA is a ribozyme.

38. A pharmaceutical composition of claim 36, wherein the pharmaceutically acceptable carrier comprises a structure which binds to a mammalian SNORF44 receptor or a mammalian SNORF44 receptor on a cell capable of being taken up by the cells after binding to the structure.

39. A pharmaceutical composition of claim 38, wherein the pharmaceutically acceptable carrier is capable of binding to a mammalian SNORF44 receptor or a mammalian SNORF44 receptor which is specific for a selected cell type.

40. A pharmaceutical composition which comprises an amount of the antibody of claim 31 effective to block binding of a ligand to a human SNORF44 receptor and a pharmaceutically acceptable carrier.

41. A transgenic, nonhuman mammal expressing DNA encoding a mammalian SNORF44 receptor of claim 1.

42. A transgenic, nonhuman mammal comprising a homologous recombination knockout of the native mammalian SNORF44 receptor.

43. A transgenic, nonhuman mammal whose genome comprises antisense DNA complementary to the DNA encoding a mammalian SNORF44 receptor of claim 1 so placed within the genome as to be transcribed into antisense mRNA which is complementary to and hybridizes with mRNA encoding the mammalian SNORF44 receptor so as to thereby reduce translation of such mRNA and expression of such receptor.

44. The transgenic, nonhuman mammal of claim 41 or 42, wherein the DNA encoding the mammalian SNORF44 receptor additionally comprises an inducible promoter.

45. A transgenic, nonhuman mammal of claim 41 or 42, wherein the transgenic, nonhuman mammal is a mouse.

46. A process for identifying a chemical compound which specifically binds to a mammalian SNORF44 receptor which comprises contacting cells containing DNA encoding, and expressing on their cell surface, the mammalian SNORF44 receptor, wherein such cells do not normally express the mammalian SNORF44 receptor, with the compound under conditions suitable for binding, and detecting specific binding of the chemical compound to the mammalian SNORF44 receptor.

47. A process for identifying a chemical compound which specifically binds to a mammalian SNORF44 receptor which comprises contacting a membrane preparation from cells containing DNA encoding, and expressing on their cell surface, the mammalian SNORF44 receptor, wherein such cells do not normally express the mammalian SNORF44 receptor, with the compound under conditions suitable for binding, and detecting specific binding of the chemical compound to the mammalian SNORF44 receptor.

48. The process of claim 46, wherein the mammalian SNORF44 receptor is a human SNORF44 receptor.

49. The process of claim 46, wherein the mammalian SNORF44 receptor has substantially the same amino acid sequence as the human SNORF44 receptor encoded by plasmid pcDNA3.1-hSNORF44-f (Patent Deposit Designation PTA-100).

50. The process of claim 46, wherein the mammalian SNORF44 receptor has substantially the same amino acid sequence as that shown in FIGS. 2A-2B (SEQ ID NO: 2).

51. The process of claim 46, wherein the mammalian SNORF44 receptor has the amino acid sequence shown in FIGS. 2A-2B (SEQ ID NO: 2).

52. The process of claim 46 or 47, wherein the compound is not previously known to bind to a mammalian SNORF44 receptor.

53. A compound identified by the process of claim 52.

54. A process of claim 46 or 47, wherein the cell is an insect cell.

55. The process of claim 46 or 47, wherein the cell is a mammalian cell.

56. The process of claim 55, wherein the mammalian cell is nonneuronal in origin.

57. The process of claim 56, wherein the nonneuronal cell is a COS-7 cell, 293 human embryonic kidney cell, a CHO cell, a NIH-3T3 cell, a mouse Y1 cell, or a LM(tk−) cell.

58. A process of claim 55, wherein the compound is a compound not previously known to bind to a mammalian SNORF44 receptor.

59. A compound identified by the process of claim 58.

60. A process involving competitive binding for identifying a chemical compound which specifically binds to a mammalian SNORF44 receptor which comprises separately contacting cells expressing on their cell surface the mammalian SNORF44 receptor, wherein such cells do not normally express the mammalian SNORF44 receptor, with both the chemical compound and a second chemical compound known to bind to the receptor, and with only the second chemical compound, under conditions suitable for binding of such compounds to the receptor, and detecting specific binding of the chemical compound to the mammalian SNORF44 receptor, a decrease in the binding of the second chemical compound to the mammalian SNORF44 receptor in the presence of the chemical compound being tested indicating that such chemical compound binds to the mammalian SNORF44 receptor.

61. A process involving competitive binding for identifying a chemical compound which specifically binds to a mammalian SNORF44 receptor which comprises separately contacting a membrane preparation from cells expressing on their cell surface the mammalian SNORF44 receptor, wherein such cells do not normally express the mammalian SNORF44 receptor, with both the chemical compound and a second chemical compound known to bind to the receptor, and with only the second chemical compound, under conditions suitable for binding of such compounds to the receptor, and detecting specific binding of the chemical compound to the mammalian SNORF44 receptor, a decrease in the binding of the second chemical compound to the mammalian SNORF44 receptor in the presence of the chemical compound being tested indicating that such chemical compound binds to the mammalian SNORF44 receptor.

62. A process of claim 60 or 61, wherein the mammalian SNORF44 receptor is a human SNORF44 receptor.

63. The process of claim 60 or 61, wherein the cell is an insect cell.

64. The process of claim 60 or 61, wherein the cell is a mammalian cell.

65. The process of claim 64, wherein the mammalian cell is nonneuronal in origin.

66. The process of claim 65, wherein the nonneuronal cell is a COS-7 cell, 293 human embryonic kidney cell, a CHO cell, a NIH-3T3 cell, a mouse Y1 cell, or a LM(tk−) cell.

67. The process of claim 60 or 61, wherein the compound is not previously known to bind to a mammalian SNORF44 receptor.

68. A compound identified by the process of claim 67.

69. A method of screening a plurality of chemical compounds not known to bind to a mammalian SNORF44 receptor to identify a compound which specifically binds to the mammalian SNORF44 receptor, which comprises
(a) contacting cells transfected with, and expressing, DNA encoding the mammalian SNORF44 receptor, or a membrane preparation of such cells, with a compound known to bind specifically to the mammalian SNORF44 receptor;
(b) contacting the cells of step (a) with the plurality of compounds not known to bind specifically to the mammalian SNORF44 receptor, under conditions permitting binding of compounds known to bind to the mammalian SNORF44 receptor;
(c) determining whether the binding of the compound known to bind to the mammalian SNORF44 receptor is reduced in the presence of the plurality of compounds, relative to the binding of the compound in the absence of the plurality of compounds; and if so
(d) separately determining the binding to the mammalian SNORF44 receptor of each compound included in the plurality of compounds, so as to thereby identify any compound included therein which specifically binds to the mammalian SNORF44 receptor.

70. A method of claim 69, wherein the mammalian SNORF44 receptor is a human SNORF44 receptor.

71. A method of claim 69, wherein the cell is a mammalian cell.

72. A method of claim 71, wherein the mammalian cell is non-neuronal in origin.

73. The method of claim 72, wherein the non-neuronal cell is a COS-7 cell, a 293 human embryonic kidney cell, a LM(tk−) cell, a CHO cell, a mouse Y1 cell, or an NIH-3T3 cell.

74. A method of detecting expression of a mammalian SNORF44 receptor by detecting the presence of mRNA coding for the mammalian SNORF44 receptor which comprises obtaining total mRNA from the cell and contacting the mRNA so obtained with the nucleic acid probe of claim 24 or 25 under hybridizing conditions, detecting the presence of mRNA hybridized to the probe, and thereby detecting the expression of the mammalian SNORF44 receptor by the cell.

75. A method of detecting the presence of a mammalian SNORF44 receptor on the surface of a cell which comprises contacting the cell with the antibody of claim 31 under conditions permitting binding of the antibody to the receptor, detecting the presence of the antibody bound to the cell, and thereby detecting the presence of the mammalian SNORF44 receptor on the surface of the cell.

76. A method of determining the physiological effects of varying levels of activity of mammalian SNORF44 receptors which comprises producing a transgenic, nonhuman mammal of claim 41 whose levels of mammalian SNORF44 receptor activity are varied by use of an inducible promoter which regulates mammalian SNORF44 receptor expression.

77. A method of determining the physiological effects of varying levels of activity of mammalian SNORF44 receptors which comprises producing a panel of transgenic, nonhuman mammals of claim 41 each expressing a different amount of mammalian SNORF44 receptor.

78. A method for identifying an antagonist capable of alleviating an abnormality wherein the abnormality is alleviated by decreasing the activity of a mammalian SNORF44 receptor comprising administering a compound to the transgenic, nonhuman mammal of claim 41, 42 or 43, and determining whether the compound alleviates any physiological and/or behavioral abnormality displayed by the transgenic, nonhuman mammal as a result of overactivity of a mammalian SNORF44 receptor, the alleviation of such an abnormality identifying the compound as an antagonist.

79. The method of claim 78, wherein the mammalian SNORF44 receptor is a human SNORF44 receptor.

80. An antagonist identified by the method of claim 78.

81. A composition comprising an antagonist of claim 80 and a carrier.

82. A method of treating an abnormality in a subject wherein the abnormality is alleviated by decreasing the activity of a mammalian SNORF44 receptor which comprises administering to the subject an effective amount of the pharmaceutical composition of claim 81, thereby treating the abnormality.

83. A method for identifying an agonist capable of alleviating an abnormality in a subject wherein the abnormality is alleviated by increasing the activity of a mammalian SNORF44 receptor comprising administering a compound to the transgenic, nonhuman mammal of claim 41, 42 or 43, and determining whether the compound alleviates any physiological and/or behavioral abnormality displayed by the transgenic, nonhuman mammal, the alleviation of such an abnormality identifying the compound as an agonist.

84. The method of claim 83, wherein the mammalian SNORF44 receptor is a human SNORF44 receptor.

85. An agonist identified by the method of claim 84.

86. A composition comprising an agonist identified by the method of claim 85 and a carrier.

87. A method of treating an abnormality in a subject wherein the abnormality is alleviated by increasing the activity of a mammalian SNORF44 receptor which comprises administering to the subject an effective amount of the composition of claim 86 so as to thereby treat the abnormality.

88. A method for diagnosing a predisposition to a disorder associated with the activity of a specific mammalian allele which comprises:
(a) obtaining DNA of subjects suffering from the disorder;
(b) performing a restriction digest of the DNA with a panel of restriction enzymes;
(c) electrophoretically separating the resulting DNA fragments on a sizing gel;
(d) contacting the resulting gel with a nucleic acid probe capable of specifically hybridizing with a unique sequence included within the sequence of a nucleic acid molecule encoding a mammalian SNORF44 receptor and labeled with a detectable marker;
(e) detecting labeled bands which have hybridized to the DNA encoding a mammalian SNORF44 receptor of claim 1 to create a unique band pattern specific to the DNA of subjects suffering from the disorder;
(f) repeating steps (a)-(e) with DNA obtained for diagnosis from subjects not yet suffering from the disorder; and
(g) comparing the unique band pattern specific to the DNA of subjects suffering from the disorder from step (e) with the band pattern from step (f) for subjects not yet suffering from the disorder so as to determine whether the patterns are the same or different and thereby diagnose predisposition to the disorder if the patterns are the same.

89. The method of claim 88, wherein a disorder associated with the activity of a specific mammalian allele is diagnosed.

90. A method of preparing the purified mammalian SNORF44 receptor of claim 10 which comprises:
(a) culturing cells which express the mammalian SNORF44 receptor;
(b) recovering the mammalian SNORF44 receptor from the cells; and
(c) purifying the mammalian SNORF44 receptor so recovered.

91. A method of preparing the purified mammalian SNORF44 receptor of claim 10 which comprises:
(a) inserting a nucleic acid encoding the mammalian SNORF44 receptor into a suitable expression vector;
(b) introducing the resulting vector into a suitable host cell;
(c) placing the resulting host cell in suitable conditions permitting the production of the mammalian SNORF44 receptor;
(d) recovering the mammalian SNORF44 receptor so produced; and optionally
(e) isolating and/or purifying the mammalian SNORF44 receptor so recovered.

92. A process for determining whether a chemical compound is a mammalian SNORF44 receptor agonist which comprises contacting cells transfected with and expressing DNA encoding the mammalian SNORF44 receptor with the compound under conditions permitting the activation of the mammalian SNORF44 receptor, and detecting any increase in mammalian SNORF44 receptor activity, so as to thereby determine whether the compound is a mammalian SNORF44 receptor agonist.

93. A process for determining whether a chemical compound is a mammalian SNORF44 receptor antagonist which comprises contacting cells transfected with and expressing DNA encoding the mammalian SNORF44 receptor with the compound in the presence of a known mammalian SNORF44 receptor agonist, under conditions permitting the activation of the mammalian SNORF44 receptor, and detecting any decrease in mammalian SNORF44 receptor activity, so as to thereby determine whether the compound is a mammalian SNORF44 receptor antagonist.

94. A process of claim 92 or 93, wherein the mammalian SNORF44 receptor is a human SNORF44 receptor.

95. A composition which comprises an amount of a SNORF44 receptor agonist determined by the process of claim 92 effective to increase activity of a mammalian SNORF44 receptor and a carrier.

96. A composition of claim 95, wherein the mammalian SNORF44 receptor agonist is not previously known.

97. A composition which comprises an amount of a mammalian SNORF44 receptor antagonist determined by the process of claim 93 effective to reduce activity of a mammalian SNORF44 receptor and a carrier.

98. A composition of claim 97, wherein the mammalian SNORF44 receptor antagonist is not previously known.

99. A process for determining whether a chemical compound specifically binds to and activates a mammalian SNORF44 receptor, which comprises contacting cells producing a second messenger response and expressing on their cell surface the mammalian SNORF44 receptor, wherein such cells do not normally express the mammalian SNORF44 receptor, with the chemical compound under conditions suitable for activation of the mammalian SNORF44 receptor, and measuring the second messenger response in the presence and in the absence of the chemical compound, a change in the second messenger response in the presence of the chemical compound indicating that the compound activates the mammalian SNORF44 receptor.

100. The process of claim 99, wherein the second messenger response comprises chloride channel activation and the change in second messenger is an increase in the level of chloride current.

101. The process of claim 99, wherein the second messenger response comprises intracellular calcium levels and the change in second messenger is an increase in the measure of intracellular calcium.

102. The process of claim 99, wherein the second messenger response comprises release of inositol phosphate and the change in second messenger is an increase in the level of inositol phosphate.

103. A process for determining whether a chemical compound specifically binds to and inhibits activation of a mammalian SNORF44 receptor, which comprises separately contacting cells producing a second messenger response and expressing on their cell surface the mammalian SNORF44 receptor, wherein such cells do not normally express the mammalian SNORF44 receptor, with both the chemical compound and a second chemical compound known to activate the mammalian SNORF44 receptor, and with only the second chemical compound, under conditions suitable for activation of the mammalian SNORF44 receptor, and measuring the second messenger response in the presence of only the second chemical compound and in the presence of both the second chemical compound and the chemical compound, a smaller change in the second messenger response in the presence of both the chemical compound and the second chemical compound than in the presence of only the second chemical compound indicating that the chemical compound inhibits activation of the mammalian SNORF44 receptor.

104. The process of claim 103, wherein the second messenger response comprises chloride channel activation and the change in second messenger response is a smaller increase in the level of chloride current in the presence of both the chemical compound and the second chemical compound than in the presence of only the second chemical compound.

105. The process of claim 103, wherein the second messenger response comprises change in intracellular calcium levels and the change in second messenger response is a smaller increase in the measure of intracellular calcium in the presence of both the chemical compound and the second chemical compound than in the presence of only the second chemical compound.

106. The process of claim 103, wherein the second messenger response comprises release of inositol phosphate and the change in second messenger response is a smaller increase in the level of inositol phosphate in the presence of both the chemical compound and the second chemical compound than in the presence of only the second chemical compound.

107. A process of any of claims 99, 100, 101 or 102, wherein the mammalian SNORF44 receptor is a human SNORF44 receptor.

108. A process of any of claims 103, 104, 105 or 106, wherein the mammalian SNORF44 receptor is a human or rat SNORF44 receptor or a human or rat SNORF44 receptor.

109. The process of any one of claims 99 or 103, wherein the cell is an insect cell.

110. The process of any one of claims 99 or 103, wherein the cell is a mammalian cell.

111. The process of claim 110, wherein the mammalian cell is nonneuronal in origin.

112. The process of claim 111, wherein the nonneuronal cell is a COS-7 cell, CHO cell, 293 human embryonic kidney cell, NIH-3T3 cell or LM(tk−) cell.

113. The process of claim 99, 100, 101 or 102, wherein the compound is not previously known to bind to a mammalian SNORF44 receptor.

114. A compound determined by the process of claim 113.

115. A composition which comprises an amount of a mammalian SNORF44 receptor agonist determined to be such by the process of claim 99, 100, 101 or 102, effective to increase activity of the mammalian SNORF44 receptor and a carrier.

116. A composition of claim 115, wherein the mammalian SNORF44 receptor agonist is not previously known.

117. A composition which comprises an amount of a mammalian SNORF44 receptor antagonist determined to be such by the process of claim 103, 104, 105 or 106, effective to reduce activity of the mammalian SNORF44 receptor and a carrier.

118. A composition of claim 117, wherein the mammalian SNORF44 receptor antagonist is not previously known.

119. A method of screening a plurality of chemical compounds not known to activate a mammalian SNORF44 receptor to identify a compound which activates the mammalian SNORF44 receptor which comprises:
(a) contacting cells transfected with and expressing the mammalian SNORF44 receptor with the plurality of compounds not known to activate the mammalian SNORF44 receptor, under conditions permitting activation of the mammalian SNORF44 receptor;
(b) determining whether the activity of the mammalian SNORF44 receptor is increased in the presence of one or more of the compounds; and if so
(c) separately determining whether the activation of the mammalian SNORF44 receptor is increased by any compound included in the plurality of compounds, so as to thereby identify each compound which activates the mammalian SNORF44 receptor.

120. A method of claim 119, wherein the mammalian SNORF44 receptor is a human SNORF44 receptor.

121. A method of screening a plurality of chemical compounds not known to inhibit the activation of a mammalian SNORF44 receptor to identify a compound which inhibits the activation of the mammalian SNORF44 receptor, which comprises:
(a) contacting cells transfected with and expressing the mammalian SNORF44 receptor with the plurality of compounds in the presence of a known mammalian SNORF44 receptor agonist, under conditions permitting activation of the mammalian SNORF44 receptor;
(b) determining whether the extent or amount of activation of the mammalian SNORF44 receptor is reduced in the presence of one or more of the compounds, relative to the extent or amount of activation of the mammalian SNORF44 receptor in the absence of such one or more compounds; and if so
(c) separately determining whether each such compound inhibits activation of the mammalian SNORF44 receptor for each compound included in the plurality of compounds, so as to thereby identify any compound included in such plurality of compounds which inhibits the activation of the mammalian SNORF44 receptor.

122. A method of claim 121, wherein the mammalian SNORF44 receptor is a human SNORF44 receptor.

123. A method of any one of claims 119, 120, 121 or 122, wherein the cell is a mammalian cell.

124. A method of claim 125, wherein the mammalian cell is non-neuronal in origin.

125. The method of claim 124, wherein the non-neuronal cell is a COS-7 cell, a 293 human embryonic kidney cell, a LM(tk−) cell or an NIH-3T3 cell.

126. A composition comprising a compound identified by the method of claim 119 or 120 in an amount effective to increase mammalian SNORF44 receptor activity and a carrier.

127. A composition comprising a compound identified by the method of claim 121 or 122 in an amount effective to decrease mammalian SNORF44 receptor activity and a carrier.

128. A method of treating an abnormality in a subject wherein the abnormality is alleviated by increasing the activity of a mammalian SNORF44 receptor which comprises administering to the subject a compound which is a mammalian SNORF44 receptor agonist in an amount effective to treat the abnormality.

129. A method of treating an abnormality in a subject wherein the abnormality is alleviated by decreasing the activity of a mammalian SNORF44 receptor which comprises administering to the subject a compound which is a mammalian SNORF44 receptor antagonist in an amount effective to treat the abnormality.

130. A process for making a composition of matter which specifically binds to a mammalian SNORF44 receptor which comprises identifying a chemical compound using the process of any one of claims 60, 61 or 69 and then synthesizing the chemical compound or a novel structural and functional analog or homolog thereof.

131. The process of claim 130, wherein the mammalian SNORF44 receptor is a human SNORF44 receptor.

132. A process for making a composition of matter which specifically binds to a mammalian SNORF44 receptor which comprises identifying a chemical compound using the process of claim 46 or 47 and then synthesizing the chemical compound or a novel structural and functional analog or homolog thereof.

133. The process of claim 132, wherein the mammalian SNORF44 receptor is a human SNORF44 receptor.

134. A process for making a composition of matter which specifically binds to a mammalian SNORF44 receptor which comprises identifying a chemical compound using the process of any of claims 92, 99 or 119 and then synthesizing the chemical compound or a novel structural and functional analog or homolog thereof.

135. The process of claim 134, wherein the mammalian SNORF44 receptor is a human SNORF44 receptor.

136. A process for making a composition of matter which specifically binds to a mammalian SNORF44 receptor which comprises identifying a chemical compound using the process of claim 103 or 121 and then synthesizing the chemical compound or a novel structural and functional analog or homolog thereof.

137. The process of claim 136, wherein the mammalian SNORF44 receptor is a human SNORF44 receptor.

138. A process for making a composition of matter which specifically binds to a mammalian SNORF44 receptor which comprises identifying a chemical compound using the process claim 93 and then synthesizing the chemical compound or a novel structural and functional analog or homolog thereof.

139. The process of claim 139, wherein the mammalian SNORF44 receptor is a human SNORF44 receptor.

140. A process for preparing a composition which comprises identifying a chemical compound by the process of any of claims 46, 47, 60, 61 or 69 or a novel structural and functional analog or homolog thereof, recovering the chemical compound free of any receptor, and then admixing a carrier and a pharmaceutically effective amount of the chemical compound.

141. The process of claim 140, wherein the mammalian SNORF44 receptor is a human SNORF44 receptor.

142. A process for preparing a composition which comprises identifying a chemical compound by the process of any of claims 92, 99 or 119 or a novel structural and functional analog or homolog thereof, recovering the chemical compound free of any receptor, and then admixing a carrier and a pharmaceutically effective amount of the chemical compound.

143. The process of claim 142, wherein the mammalian SNORF44 receptor is a human SNORF44 receptor.

144. A process for preparing a composition which comprises identifying a chemical compound by the process of any of claims 93, 103 or 121 or a novel structural and functional analog or homolog thereof, recovering the chemical compound free of any receptor, and then admixing a carrier and a pharmaceutically effective amount of the chemical compound.

145. The process of claim 144, wherein the mammalian SNORF44 receptor is a human SNORF44 receptor.
